(19) United States
(12) Patent Application Publication
(10) Pub. No.: US 2024/0301469 A1
(43) Pub. Date: Sep. 12, 2024

(54) MODIFYING, SEPARATING AND DETECTING PROTEOFORMS

(71) Applicant: Nautilus Subsidiary, Inc., Seattle, WA (US)

(72) Inventors: Maureen NEWMAN, Redwood City, CA (US); Jarrett D. EGERTSON, Rancho Palos Verdes, CA (US); James Henry JOLY, San Mateo, CA (US); Michael Agusto DARCY, Fremont, CA (US)

(21) Appl. No.: 18/437,580

(22) Filed: Feb. 9, 2024

Related U.S. Application Data
(60) Provisional application No. 63/486,747, filed on Feb. 24, 2023.

(51) Int. Cl. C12Q 1/6809; C12Q 1/6816

(52) U.S. Cl. CPC C12Q 1/6809; C12Q 1/6816

(57) ABSTRACT

Compositions and methods for detecting, characterizing and identifying proteoforms including, for example, determining presence or absence of a particular post-translational modification or a post-translational modification of a particular amino acid in a proteoform, and determining the location(s) of one or more post-translational modifications in the amino acid sequence of a proteoform.

### CROSS-REFERENCE TO RELATED APPLICATIONS

This application claims priority to U.S. Provisional Application No. 63/486,747, filed on Feb. 24, 2023, which application is incorporated herein by reference in its entirety.

### SEQUENCE LISTING

The instant application contains a Sequence Listing which has been submitted electronically in XML format and is hereby incorporated by reference in its entirety. The XML copy, created on Feb. 7, 2024, is named NBIOT.019SeqListing.xml and is 2,343 bytes in size.

### BACKGROUND

Medical research and clinical diagnostics have been revolutionized by the emergence of high throughput technology platforms that routinely decode the human genome or human transcriptome in a matter of hours. An individual's genome provides a set of instructions for development, behavior, risk of disease, responsiveness to therapeutic treatments, longevity and many other characteristics. As such, the genome provides a powerful source for evaluating risk and predicting outcomes to certain treatments or medications. An individual's transcriptome is the collection of RNA transcripts that are expressed from the genome. The RNA transcripts are, in turn, translated into proteins, the proteins being the workhorses that perform the biological functions instructed by the genome. High throughput tools allow characterization and quantification of the transcriptome and, in some cases, clinically relevant diagnoses or prognoses can be made. However, in many cases, a transcriptome does not provide adequate diagnostic or prognostic precision to guide treatment. This is because the collection of proteins (i.e. the proteome) that is present in a biological system at any given time is not a direct reflection of the transcriptome. The number and types of proteins present at any given time is also influenced by processes that degrade or remove proteins. These are dynamic processes that are exquisitely responsive to prevailing conditions and complex processes that are variably applied to different proteins.

Moreover, protein activity is regulated not merely by the amount and types of proteins that are present, but also by the number and type of chemical modifications that are made to the proteins. These so-called post-translational modifications act as positive and negative regulators of protein activity and are dynamically responsive to conditions experienced by the individual at any given time. There exists a need for proteome-scale characterization of biological systems, thereby further advancing the revolution in medical research and clinical diagnostics. The compositions, methods and apparatus of the present disclosure satisfy this need and provide related advantages as well.

### SUMMARY

The present disclosure provides a method of modifying proteoforms, including (a) providing a first aliquot from a biological sample and a second aliquot from the biological sample, wherein a plurality of different proteoforms that are present in the first aliquot is also present in the second aliquot, wherein the proteoforms have post-translationally modified amino acids and standard amino acids; (b) treating proteoforms of the first aliquot by: (i) chemically modifying the standard amino acids, thereby forming artificially modified amino acids, (ii) then removing post-translational modifications from the post-translationally modified amino acids, thereby forming standard amino acids, (iii) then attaching the proteoforms of the first aliquot to a label or solid support; and (c) treating proteoforms of the second aliquot by attaching the proteoforms of the second aliquot to a label or solid support.

In some configurations, a method of modifying proteoforms can include (a) providing a first aliquot from a biological sample and a second aliquot from the biological sample, wherein a plurality of different proteoforms that are present in the first aliquot is also present in the second aliquot, wherein the proteoforms have post-translationally modified amino acids and standard amino acids; (b) treating proteoforms of the first aliquot by: (i) chemically modifying the standard amino acids, thereby forming artificially modified amino acids, (ii) then removing post-translational modifications from the post-translationally modified amino acids, thereby forming standard amino acids, (iii) then attaching the proteoforms of the first aliquot to a first set of unique identifiers; and (c) treating proteoforms of the second aliquot by attaching the proteoforms of the second aliquot to a second set of unique identifiers.

A method of the present disclosure can be configured to detect proteins such as proteoforms. For example, a method set forth herein for modifying proteoforms can include a step of detecting proteoforms. Accordingly, the present disclosure provides a method of detecting proteoforms, including (a) providing a first aliquot from a biological sample and a second aliquot from the biological sample, wherein a plurality of different proteoforms that are present in the first aliquot is also present in the second aliquot, wherein the proteoforms have post-translationally modified amino acids and standard amino acids; (b) treating proteoforms of the first aliquot by: (i) chemically modifying the standard amino acids, thereby forming artificially modified amino acids, (ii) then removing post-translational modifications from the post-translationally modified amino acids, thereby forming standard amino acids, (iii) then attaching the proteoforms of the first aliquot to a label, particle, solid support, or unique identifier, and (iv) detecting proteoforms of the first aliquot; (c) treating proteoforms of the second aliquot by (i) attaching the proteoforms of the second aliquot to a label, particle, solid support, or unique identifier, and (ii) detecting proteoforms of the second aliquot.

The present disclosure further provides a method of detecting proteoforms, including steps of: (a) providing a first aliquot from a biological sample and a second aliquot from the biological sample, wherein a plurality of different proteoforms that are present in the first aliquot is also present in the second aliquot, wherein the proteoforms have post-translationally modified amino acids and standard amino acids; (b) treating proteoforms of the first aliquot by: (i) chemically modifying the standard amino acids, thereby forming artificially modified amino acids, (ii) then removing post-translational modifications from the post-translationally modified amino acids, thereby forming standard amino acids, and (iii) then detecting proteoforms of the first aliquot; (c) detecting proteoforms of the second aliquot; and (d) identifying presence of a post-translational modification in a proteoform of the biological sample based on differential detection of a proteoform in the first aliquot and in the second aliquot.

A method of detecting a proteoform can include steps of (a) contacting an array of proteoforms with a first affinity reagent and a second affinity reagent, thereby forming a complex including the first affinity reagent bound to a post-translationally modified amino acid in a proteoform at an address of the array and the complex further including the second affinity reagent bound to an epitope in the proteoform, wherein the first affinity reagent is attached to a first nucleic acid and the second affinity reagent is attached to a second nucleic acid; (b) contacting the complex with a splint nucleic acid, wherein a first nucleotide sequence region of the splint nucleic acid hybridizes to the first nucleic acid and a second nucleotide sequence region of the splint nucleic acid hybridizes to the second nucleic acid; (c) detecting hybridization of the first and second nucleic acids to the splint nucleic acid, thereby detecting the proteoform at the address. Optionally, the epitope can be a sequence of standard amino acids or an artificially modified amino acid.

A method of detecting a proteoform, can include steps of (a) contacting an array of proteoforms with a multivalent affinity reagent, thereby forming a complex including a first affinity moiety of the multivalent affinity reagent bound to a post-translationally modified amino acid of a proteoform at an address of the array, the complex further including a second affinity moiety of the multivalent affinity reagent bound to an epitope (e.g. a sequence of standard amino acids, an artificial moiety or another post-translationally modified amino acid) in the proteoform; and (b) detecting the complex at the address.

### BRIEF DESCRIPTION OF DRAWINGS

FIG. 1A shows a diagram of a method for artificially modifying standard amino acids of proteoforms, removing post-translational moieties from the proteoforms and attaching the proteoforms to an array via artificially modified amino acids.

FIG. 1B shows a diagram of a method for attaching proteoforms to an array via standard amino acids.

FIG. 1C shows a diagram of a method for artificially modifying standard amino acids of proteoforms and attaching the proteoforms to an array via artificially modified amino acids.

FIG. 1D shows a diagram of a method for artificially modifying standard amino acids of proteoforms and attaching the proteoforms to an array via post-translationally modified amino acids.

FIG. 1E shows a diagram of a method for attaching proteoforms to an array via standard amino acids and modifying the attached proteoforms to remove post-translational modifications.

FIG. 2A shows a diagram of a method for blocking standard amino acids of proteoforms, removing post-translational moieties from the proteoforms and attaching the proteoforms to an array via standard amino acids.

FIG. 2B shows a diagram of a method for attaching proteoforms to an array via standard amino acids.

FIG. 2C shows a diagram of a method for blocking standard amino acids of proteoforms and attaching the proteoforms to an array via post-translational moieties of amino acids.

FIG. 2D shows a diagram of a method for blocking standard amino acids of proteoforms and attaching the proteoforms to an array via artificial moieties of amino acids.

FIG. 3 shows a diagram of a method for differential modification of proteoforms within a sample.

FIG. 4 shows steps of a proximity assay for detecting a protein having a post-translational moiety and a peptide epitope.

FIG. 5A shows binding of a first affinity moiety of a multivalent affinity reagent to an acetylated lysine in a protein and a second affinity moiety of the affinity reagent to trimer of standard amino acids (i.e. TSN) in the protein.

FIG. 5B shows binding of a monovalent affinity reagent (e.g. the first affinity moiety of FIG. 5A), to the acetylated lysine in the protein of FIG. 5A.

FIG. 5C shows binding of a monovalent affinity reagent (e.g. the second affinity moiety of FIG. 5A), to the TSN epitope in the protein of FIG. 5A.

### DETAILED DESCRIPTION

The present disclosure provides compositions and methods that are useful for detecting, characterizing and identifying proteoforms. For example, the presence or absence of a particular post-translational modification or a particular post-translationally modified amino acid can be determined. In some embodiments, a proteoform can be characterized with respect to the location(s) of one or more post-translational modifications in the amino acid sequence of the proteoform. Locations can be identified, for example, at a specific position of the amino acid sequence for the proteoform. However, in some cases, the location of a post-translational modification in a proteoform can be determined relative to a particular structural motif of the proteoform. For example, a post-translational moiety of a proteoform can be located relative to a short sequence of amino acids in the proteoform or relative to another post-translational moiety in the proteoform.

In a particular aspect, a method of the present disclosure can be configured to differentially modify one or more proteoforms of a first aliquot from a biological sample compared to copies of the one or more proteoforms in a second aliquot from the biological sample. The differential modifications can target standard versions of amino acids and/or post-translationally modified versions of amino acids. The differentially modified proteoforms can be detected, for example, using affinity reagents that specifically recognize and bind the modifications. For example, the affinity reagents can selectively recognize and bind a standard version of a given amino acid compared to a post-translationally modified version of the amino acid, or vice versa. In another example, proteoforms can be differentially modified to include artificial moieties on standard amino acids or on post-translationally modified amino acids. In this scenario, affinity reagents can be used that selectively recognize or bind one of the following three versions of a given amino acid: (1) a standard version of the amino acid, (2) a post-translationally modified version of the amino acid or (3) an artificially modified version of the amino acid, compared to the other two of the three versions of the amino acid.

In another aspect, a method of the present disclosure can be configured to determine the relative proximity of two or more epitopes in a protein based on proximity of two or more affinity reagents when bound to the epitopes in the protein. For example, the protein can be a particular proteoform and at least one of the affinity reagents can bind an epitope having a post-translational modification. The location of the post-translational modification can be determined relative to the location of another post-translational modification that binds a second affinity reagent or relative to the location of a sequence of standard amino acids (e.g. a dimer, trimer, tetramer or pentamer). Such methods can also be used to locate an artificially modified amino acid relative to the location of another artificially modified amino acid, relative to a post-translationally modified amino acid or relative to the location of a sequence of standard amino acids.

In yet another aspect, a method of this disclosure can be configured to detect a protein using a multivalent affinity reagent. For example, the multivalent affinity reagent can have a first affinity moiety that recognizes a first epitope in the protein and a second affinity moiety that recognizes a second epitope in the protein. Binding of the multivalent affinity reagent can be used to determine the presence of both epitopes in the protein and to determine that the epitopes are located within a given distance of each other in accordance with the maximum distance between the two affinity moieties in the multivalent affinity reagent. A multivalent affinity reagent can include (1) a first affinity moiety that recognizes or binds to first epitope, such as a post-translationally modified amino acid, artificially modified amino acid or standard amino acid and (2) a second affinity moiety that recognizes or binds to a second epitope, such as a post-translationally modified amino acid, a sequence of standard amino acids, or an artificially modified amino acid.

Methods and compositions set forth herein are well suited for use in combination with other protein assays. For example, a binding assay can be configured to use a plurality of affinity reagents, a binding model, a database of candidate proteins and a trained algorithm to identify one or more proteins. In some cases, it can be difficult to determine if a particular affinity reagent is observed to bind (or not bind) a given protein due to the protein containing a post-translational modification in the epitope for the affinity reagents. This can be all the more difficult in the context of a single-molecule assays wherein detection events can appear stochastic in nature. Methods and compositions for detecting and locating post-translational modifications can be useful for improving accuracy of, or confidence in, results obtained from a single molecule assay, such as a single molecule binding assay or single molecule amino acid sequencing assay. Detecting or locating post-translational modifications can aid in understanding results when assay reagents are known or suspected of being sensitive to the presence or absence of post-translational modifications.

Terms used herein will be understood to take on their ordinary meaning in the relevant art unless specified otherwise. Several terms used herein and their meanings are set forth below.

As used herein, the term “address” refers to a location in an array where a particular analyte (e.g. protein, or proteoform) is present. An address can contain a single analyte (i.e. one and only one analyte), or it can contain a population of several analytes of the same species (i.e. an ensemble of the analyte species). Alternatively, an address can include a population of different analytes. Addresses are typically discrete. The discrete addresses can be contiguous, or they can be separated by interstitial spaces. An array useful herein can have, for example, addresses that are separated by less than 100 microns, 10 microns, 1 micron, 100 nm, 10 nm or less. Alternatively or additionally, an array can have addresses that are separated by at least 10 nm, 100 nm, 1 micron, 10 microns, or 100 microns. The addresses can each have an area of less than 1 square millimeter, 500 square microns, 100 square microns, 10 square microns, 1 square micron, 100 square nm or less. An array can include at least about 1×10⁴, 1×10⁵, 1×10⁶, 1×10⁸, 1×10¹⁰, 1×10¹², 1×10¹⁴, or more addresses.

As used herein, the term “affinity moiety” refers to a moiety of a molecule or other substance, the moiety being capable of specifically or reproducibly binding to an analyte (e.g. protein). An affinity moiety can be larger than, smaller than, or the same size as the analyte. An affinity moiety may form a reversible or irreversible bond with an analyte. An affinity moiety may bind with an analyte in a covalent or non-covalent manner. An affinity moiety may be reactive (e.g. SpyTag or Spycatcher), catalytic (e.g., kinase, protease, etc.) or non-reactive (e.g., antibody or functional fragment thereof). An affinity moiety can be non-reactive and non-catalytic, thereby not permanently altering the chemical structure of an analyte to which it binds. A particularly useful affinity moiety is an antibody, such as a full-length antibody or functional fragment thereof. A functional fragment of an antibody can include any fragment that is capable of binding to an epitope with a detectable affinity, such as a Fab, Fab′, F(ab′)₂, single-chain variable (scFv), di-scFv, tri-scFv, or microantibody. Other useful affinity moieties include, for example, affibodies, affilins, affimers, affitins, alphabodies, anticalins, avimers, DARPins, monobodies, nanoCLAMPs, nucleic acid aptamers, polypeptide aptamers, lectins or functional fragments thereof. An “affinity reagent” is a molecule or other entity having at least one affinity moiety.

As used herein, the term “array” refers to a population of analytes (e.g. proteins) that are associated with unique identifiers such that the analytes can be distinguished from each other. A unique identifier can be, for example, a solid support (e.g. particle or bead), address on a solid support, tag, label (e.g. luminophore), particle (e.g. structured nucleic acid particle) or barcode (e.g. nucleic acid barcode) that is associated with an analyte and that is distinct from other identifiers in the array. Analytes can be associated with unique identifiers by attachment, for example, via covalent bonds or non-covalent bonds (e.g. ionic bond, hydrogen bond, van der Waals forces, electrostatics etc.). An array can include different analytes that are each attached to different unique identifiers. An array can include different unique identifiers that are attached to the same or similar analytes. An array can include separate solid supports or separate addresses that each bear a different analyte, wherein the different analytes can be identified according to the locations of the solid supports or addresses.

As used herein, the term “attached” refers to the state of two things being joined, fastened, adhered, connected or bound to each other. Attachment can be covalent or non-covalent. For example, a label can be attached to a protein by a covalent or non-covalent bond. A covalent bond is characterized by the sharing of pairs of electrons between atoms. A non-covalent bond is a chemical bond that does not involve the sharing of pairs of electrons and can include, for example, hydrogen bonds, ionic bonds, van der Waals forces, hydrophilic interactions, adhesion, adsorption, and hydrophobic interactions.

As used herein, the term “artificial” when used in reference to a substance, means that the substance is made by human activity rather than occurring naturally. The substance can be a moiety of a molecule, such as an amino acid of a protein. For example, an amino acid that is chemically modified at least in part by human activity is referred to as an “artificial amino acid.” An amino acid that is chemically modified to add a moiety is said to have an “artificial moiety”.

As used herein the term “blocked,” when used in reference to a substance or moiety, refers to the substance or moiety having been modified to prevent the substance or moiety from participating in a reaction in which the non-modified version of the substance or moiety was capable of participating. For example, an amino acid is referred to as being blocked when a reactive moiety in the side chain of the amino acid is modified to prevent the reactive moiety from further reaction. An amino acid of a protein can be permanently blocked, for example, by incorporation of a blocking moiety that cannot be removed without destroying the amino acid or the protein, or by removal of a reactive moiety from the amino acid. Alternatively, an amino acid of a protein can be reversibly blocked, for example, by incorporation of a blocking moiety that can be removed without destroying the amino acid or the protein.

As used herein, the term “click reaction” refers to single-step, thermodynamically-favorable conjugation reaction utilizing biocompatible reagents. A click reaction may be configured to not utilize toxic or biologically incompatible reagents (e.g., acids, bases, heavy metals) or to not generate toxic or biologically incompatible byproducts. A click reaction may utilize an aqueous solvent or buffer (e.g., phosphate buffer solution, Tris buffer, saline buffer, MOPS, etc.). A click reaction may be thermodynamically favorable if it has a negative Gibbs free energy of reaction, for example a Gibbs free energy of reaction of less than about −5 kiloJoules/mole (KJ/mol), −10 KJ/mol, −25 KJ/mol, −100 KJ/mol, −250 kJ/mol, −500 KJ/mol, or less. Exemplary click reactions may include metal-catalyzed azide-alkyne cycloaddition, strain-promoted azide-alkyne cycloaddition (SPAAC), strain-promoted azide-nitrone cycloaddition, strained alkene reactions, thiol-ene reaction, photo thiol-ene reaction, Diels-Alder reaction, inverse electron demand Diels-Alder reaction (IEDDA), [3+2] cycloaddition, [4+1] cycloaddition, nucleophilic substitution, dihydroxylation, thiol-yne reaction, photo thiol-yne reaction, photoclick reaction, nitrone dipole cycloaddition, norbornene cycloaddition, oxanobornadiene cycloaddition, tetrazine ligation, Staudinger ligation and tetrazole photoclick reactions. Exemplary reactive moieties utilized to perform click reactions may include alkenes, alkynes, azides, epoxides, amines, thiols, nitrones, isonitriles, isocyanides, aziridines, activated esters, and tetrazines. Other well-known click conjugation reactions may be used having complementary bioorthogonal reaction species, for example, where a first click component comprises a hydrazine moiety and a second click component comprises an aldehyde or ketone group, and where the product of such a reaction comprises a hydrazone functional group or equivalent. Exemplary bioorthogonal and click reactions are set forth in US Pat. App. Pub. No. 2021/0101930 A1, which is incorporated herein by reference.

The term “comprising” is intended herein to be open-ended, including not only the recited elements, but further encompassing any additional elements.

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, the term “epitope” refers to an affinity target within a protein or other analyte. Epitopes may include amino acid sequences that are sequentially adjacent in the primary structure of a protein. Epitopes may include amino acids that are structurally adjacent in the secondary, tertiary or quaternary structure of a protein despite being non-adjacent in the primary structure of the protein. An epitope can be, or can include, a post-translational moiety of a protein, such as a phosphate of a phosphotyrosine, phosphoserine, phosphothreonine, or phosphohistidine. An epitope can optionally be recognized by or bound to an antibody. However, an epitope need not necessarily be recognized by any antibody, for example, instead being recognized by an aptamer, mini-protein or other affinity reagent. An epitope can optionally bind an antibody to elicit an immune response. However, an epitope need not necessarily participate in, nor be capable of, eliciting an immune response.

As used herein, the term “fluid-phase,” when used in reference to a molecule, means the molecule is in a state wherein it is mobile in a fluid, for example, being capable of diffusing through the fluid.

As used herein, the term “immobilized,” when used in reference to a molecule that is in contact with a fluid phase, refers to the molecule being prevented from diffusing in the fluid phase. For example, immobilization can occur due to the molecule being confined at, or attached to, a solid phase. Immobilization can be temporary (e.g. for the duration of one or more steps of a method set forth herein) or permanent. Immobilization can be reversible or irreversible under conditions utilized for a method, system or composition set forth herein.

As used herein, the term “label” refers to a molecule or moiety that provides a detectable characteristic. The detectable characteristic can be, for example, an optical signal such as absorbance of radiation, luminescence emission, luminescence lifetime, luminescence polarization, fluorescence emission, fluorescence lifetime, fluorescence polarization, or the like; Rayleigh and/or Mie scattering; binding affinity for a ligand or receptor; magnetic properties; electrical properties; charge; mass; radioactivity or the like. Exemplary labels include, without limitation, a luminophore (e.g. fluorophore), chromophore, nanoparticle (e.g., gold, silver, carbon nanotubes, quantum dots, upconversion nanocrystals), heavy atoms, radioactive isotope, mass label, charge label, spin label, receptor, ligand, or the like. A label may produce a signal that is detectable in real-time (e.g., fluorescence, luminescence, radioactivity). A label may produce a signal that is detected off-line (e.g., a nucleic acid barcode) or in a time-resolved manner (e.g., time-resolved fluorescence). A label may produce a signal with a characteristic frequency, intensity, polarity, duration, wavelength, sequence, or fingerprint.

As used herein, the term “moiety” refers to a component or part of a molecule. The term does not necessarily denote the relative size of the component or part compared to the rest of the molecule, unless indicated otherwise. A moiety can include one or more atoms.

As used herein, the term “monovalent” when used in reference to an affinity reagent, means that the affinity reagent contains one, and only one, affinity moiety, or that the affinity reagent contains multiple affinity moieties having the same affinity for a given epitope or having affinity for the same epitopes. Typically, multiple affinity moieties of a monovalent affinity reagent will be structurally identical to each other.

As used herein, the term “multivalent” when used in reference to an affinity reagent, means that the affinity reagent contains two or more affinity moieties having different affinity for a given epitope, or having affinity for different epitopes, respectively. Typically, multiple affinity moieties of a multivalent affinity reagent will be structurally different from each other.

As used herein, the term “nucleic acid origami” refers to a nucleic acid construct having an engineered tertiary or quaternary structure. A nucleic acid origami may include DNA, RNA, PNA, modified or non-natural nucleic acids, or combinations thereof. A nucleic acid origami may include a plurality of oligonucleotides that hybridize via sequence complementarity to produce the engineered structure of the origami. A nucleic acid origami may include sections of single-stranded or double-stranded nucleic acid, or combinations thereof. Exemplary nucleic acid origami structures may include nanotubes, nanowires, cages, tiles, nanospheres, blocks, and combinations thereof. A nucleic acid origami can optionally include a relatively long scaffold nucleic acid to which multiple smaller nucleic acids hybridize, thereby creating folds and bends in the scaffold that produce an engineered structure. The scaffold nucleic acid can be circular or linear. The scaffold nucleic acid can be single stranded but for hybridization to the smaller nucleic acids. A smaller nucleic acid (sometimes referred to as a “staple”) can hybridize to two regions of the scaffold, wherein the two regions of the scaffold are separated by an intervening region that does not hybridize to the smaller nucleic acid.

As used herein, the term “post-translational modification” refers to a naturally occurring change to the chemical composition of a protein compared to the chemical composition encoded by the gene for the protein. Exemplary changes include those that alter the presence, absence or relative arrangement of different regions of amino acid sequence (e.g., splicing variants, or protein processing variants of a single gene), or due to presence or absence of different moieties on particular amino acids (e.g., post-translationally modified variants of a single gene). A post-translational modification can be derived from an in vivo process or in vitro process. Exemplary post-translational modifications include those classified by the PSI-MOD ontology. See Smith, L. M. et al. Nat. Methods, 2013, 10, 186-187, which is incorporated herein by reference.

As used herein, the term “protein” refers to a molecule comprising two or more amino acids joined by a peptide bond. A protein may also be referred to as a polypeptide, oligopeptide, or peptide. A protein can be a naturally-occurring molecule, or synthetic molecule. A protein may include one or more non-natural amino acids, modified amino acids, or non-amino acid linkers. A protein may contain D-amino acid enantiomers, L-amino acid enantiomers or both. Amino acids of a protein may be modified naturally or synthetically, such as by post-translational modifications. In some circumstances, different proteins may be distinguished from each other based on different genes from which they are expressed in an organism, different primary sequence length or different primary sequence composition. Proteins expressed from the same gene may nonetheless be different proteoforms, for example, being distinguished based on non-identical length, non-identical amino acid sequence or non-identical post-translational modifications. Different proteins can be distinguished based on one or both of gene of origin and proteoform state.

As used herein, the term “single,” when used in reference to an object such as an analyte, means that the object is individually manipulated or distinguished from other objects. A single analyte can be a single molecule (e.g. single protein), a single complex of two or more molecules (e.g. a multimeric protein having two or more separable subunits, a single protein attached to a structured nucleic acid particle or a single protein attached to an affinity reagent), a single particle, or the like. Reference herein to a “single analyte” in the context of a composition, system or method herein does not necessarily exclude application of the composition, system or method to multiple single analytes that are manipulated or distinguished individually, unless indicated contextually or explicitly to the contrary.

As used herein, the term “single-analyte resolution” refers to the detection of, or ability to detect, an analyte on an individual basis, for example, as distinguished from its nearest neighbor in an array of analytes.

As used herein, the term “solid support” refers to a substrate that is insoluble in aqueous liquid. Optionally, the substrate can be rigid. The substrate can be non-porous or porous. The substrate can optionally be capable of taking up a liquid (e.g. due to porosity) but will typically, but not necessarily, be sufficiently rigid that the substrate does not swell substantially when taking up the liquid and does not contract substantially when the liquid is removed by drying. A nonporous solid support is generally impermeable to liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor™, silica or silica-based materials including silicon and modified silicon, carbon, metals, inorganic glasses, optical fiber bundles, gels, and polymers. In particular configurations, a flow cell contains the solid support such that fluids introduced to the flow cell can interact with a surface of the solid support to which one or more components of a binding event (or other reaction) is attached.

As used herein, the term “standard,” when used in reference to amino acids in a protein, refers to amino acids that are encoded directly by the codons of the universal genetic code. Amino acids having post-translational moieties or artificial moieties are not considered standard amino acids, even if the amino acids were derived from standard amino acids.

As used herein, the term “structured nucleic acid particle” or “SNAP” refers to a single- or multi-chain polynucleotide molecule having a compacted three-dimensional structure. The compacted three-dimensional structure can optionally be characterized in terms of hydrodynamic radius or Stoke's radius of the SNAP relative to a random coil or other non-structured state for a nucleic acid having the same mass or sequence length as the SNAP. The compacted three-dimensional structure can optionally be characterized with regard to tertiary structure. For example, a SNAP can be configured to have an increased number of internal binding interactions between regions of a polynucleotide strand, less distance between the regions, increased number of bends in the strand, and/or more acute bends in the strand, as compared to a nucleic acid molecule of similar length in a random coil or other non-structured state. Alternatively or additionally, the compacted three-dimensional structure can optionally be characterized with regard to tertiary or quaternary structure. For example, a SNAP can be configured to have an increased number of interactions between polynucleotide strands or less distance between the strands, as compared to a nucleic acid molecule of similar length in a random coil or other non-structured state. In some configurations, the secondary structure of a SNAP can be configured to be more dense than a nucleic acid molecule of similar length in a random coil or other non-structured state. A SNAP may contain DNA, RNA, PNA, modified or non-natural nucleic acids, or combinations thereof. A SNAP may include a plurality of oligonucleotides that hybridize to form the SNAP structure. The plurality of oligonucleotides in a SNAP may include oligonucleotides that are attached to other molecules (e.g., probes, analytes such as polypeptides, reactive moieties, or detectable labels) or are configured to be attached to other molecules (e.g., by functional groups). A SNAP may include engineered or rationally designed structures. Exemplary SNAPs include nucleic acid origami and nucleic acid nanoballs.

As used herein, the term “unique identifier” refers to a moiety, object or substance that is associated with an analyte and that is distinct from other identifiers, throughout one or more steps of a process. The moiety, object or substance can be, for example, a solid support such as a particle or bead; a location on a solid support; a spatial address in an array; a tag; a label such as a luminophore; a molecular barcode such as a nucleic acid having a unique nucleotide sequence or a protein having a unique amino acid sequence; or an encoded device such as a radiofrequency identification (RFID) chip, electronically encoded device, magnetically encoded device or optically encoded device. The process in which a unique identifier is used can be an analytical process, such as a method for detecting, identifying, characterizing or quantifying an analyte; a separation process in which at least on analyte is separated from other analytes; or a synthetic process in which an analyte is modified or produced. The unique identifier can be associated with an analyte via immobilization. For example, a unique identifier can be covalently or non-covalently (e.g. ionic bond, hydrogen bond, van der Waals forces etc.) attached to an analyte. A unique identifier can be exogenous to an associated analyte, for example, being synthetically attached to the associated analyte. Alternatively, a unique identifier can be endogenous to the analyte, for example, being attached or associated with the analyte in the native milieu of the analyte.

As used herein, the term “unique identifier label” refers to a unique identifier that is a particle, molecule or moiety that provides a detectable characteristic. The detectable characteristic can be, for example, an optical signal such as absorbance of radiation, luminescence (e.g. fluorescence) emission, luminescence lifetime, luminescence polarization, or the like; Rayleigh and/or Mie scattering; binding affinity for a ligand or receptor; magnetic properties; electrical properties; charge; mass; radioactivity or the like. Exemplary labels include, without limitation, a fluorophore, luminophore, chromophore, nanoparticle (e.g., gold, silver, carbon nanotubes), heavy atoms, radioactive isotope, mass label, charge label, spin label, receptor, ligand, or the like.

The embodiments set forth below and recited in the claims can be understood in view of the above definitions.

### Differential Capture or Labelling of Proteoforms

The present disclosure provides a method of modifying proteoforms, including (a) providing a first aliquot from a biological sample and a second aliquot from the biological sample, wherein a plurality of different proteoforms that are present in the first aliquot is also present in the second aliquot, wherein the proteoforms have post-translationally modified amino acids and standard amino acids; (b) treating proteoforms of the first aliquot by: (i) chemically modifying the standard amino acids, thereby forming artificially modified amino acids, (ii) then removing post-translational modifications from the post-translationally modified amino acids, thereby forming standard amino acids, (iii) then attaching the proteoforms of the first aliquot to a label or solid support; and (c) treating proteoforms of the second aliquot by attaching the proteoforms of the second aliquot to a label or solid support.

In some configurations, a method of modifying proteoforms can include (a) providing a first aliquot from a biological sample and a second aliquot from the biological sample, wherein a plurality of different proteoforms that are present in the first aliquot is also present in the second aliquot, wherein the proteoforms have post-translationally modified amino acids and standard amino acids; (b) treating proteoforms of the first aliquot by: (i) chemically modifying the standard amino acids, thereby forming artificially modified amino acids, (ii) then removing post-translational modifications from the post-translationally modified amino acids, thereby forming standard amino acids, (iii) then attaching the proteoforms of the first aliquot to a first set of unique identifiers; and (c) treating proteoforms of the second aliquot by attaching the proteoforms of the second aliquot to a second set of unique identifiers.

A method of the present disclosure can include a step of providing a first aliquot from a biological sample and a second aliquot from the biological sample. Typically, the first and second aliquots will have apparently equivalent representations of the protcome of the biological sample. For example, a plurality of different proteoforms that is present in the first aliquot can also be present in the second aliquot. The aliquots can be apparently equivalent with regard to the total quantity or concentration of proteins in each aliquot, the quantity or concentration of a given genetically encoded protein in each aliquot, the relative amounts or concentrations of different genetically encoded proteins in one aliquot compared to the other, the quantity or concentration of a given proteoform in each aliquot, and/or the relative amounts or concentrations of different proteoforms in one aliquot compared to the other. Two or more aliquots can be obtained from a biological sample under similar conditions and the aliquots can be treated similarly prior to one or more steps of a method set forth herein. As such, one aliquot can serve as a control for treatments administered to the other aliquot in a method set forth herein.

In some cases, two or more aliquots can be obtained from a sample using different conditions, respectively. Alternatively or additionally, two or more aliquots that are obtained from a given sample can be treated differently. Differences in how the aliquots are obtained or treated can be evaluated using methods set forth herein, for example, by comparing results obtained for proteoforms in different aliquots.

A method set forth herein can include a step of chemically modifying a standard version of an amino acid, thereby forming an artificially modified version of the amino acid. For example, a standard amino acid can be modified by addition of an artificial moiety. Reactive moieties of amino acids include, for example, (1) an amine that is present at the amino terminus of a polypeptide or in the side chain of a lysine, histidine or arginine; (2) a sulfur that is present in the side chain of a cysteine or methionine; (3) a carboxyl that is present at the carboxy terminus of a polypeptide or in the side chain of an aspartic acid or glutamic acid; (4) an oxygen that is present in the side chain of a serine, threonine or tyrosine; or (5) an amide that is present in the side chain of a glutamine or asparagine. A protein can include a plurality of amino acids of a given type that are reactive, such as those identified above.

In some configurations of the methods set forth herein, an artificial moiety that is present in an artificially modified amino acid is a blocking moiety. A blocking moiety when present in one or more amino acids of a protein can function to prevent the blocked amino acid(s) from participating in a reaction that is intended for other amino acids in the protein. In some cases, the blocking moieties can be selected from post-translational moieties that are found in nature (e.g. one or more of those set forth herein) or derivatives thereof. Such moieties can be added artificially, for example, using enzymes that target particular amino acids in vitro, or using synthetic chemistry approaches. In some cases, a blocking moiety is reactive, but its reactivity is orthogonal to a reaction that is carried out for other amino acids. As such, standard amino acids in a given protein can be artificially modified by addition of an attachment moiety that has orthogonal reactivity with an attachment moiety added to another amino acid of the given protein. A variety of reagents have been developed for modifying proteins using click chemistry. For example, azide and alkyne carbohydrate species are set forth in Parker and Pratt, Cell 180:605-632 (2020), which is incorporated herein by reference. Another useful blocking chemistry uses the Protein Methyl-tetrazine-CDM Tag (ProMTag) that includes a carboxyl dimethyl maleic anhydride (CDM) moiety that reversibly reacts with protein primary amines and a methyl tetrazine moiety that reacts irreversibly with transcyclooctene moieties of blocking reagents. See Biedka et al., J. Proteome Res. 20:4787-4800 (2021), which is incorporated herein by reference.

A particularly useful artificial moiety is an attachment moiety. An attachment moiety can provide a reactive moiety that is capable of bonding, covalently or non-covalently, with a substance or material of interest. As set forth in further detail herein, the attachment moiety can be reactive with a partner moiety of a particle (e.g. structured nucleic acid particle or solid-support particle), solid support, gel, label, unique identifier (e.g. address of an array), or blocking moiety.

Click moieties, or other moieties used in bioorthogonal reactions, can be particularly useful as attachment moieties. Such moieties are capable of creating covalent attachments via click reactions. Exemplary click reactions include, but are not limited to, amide formation reaction, reductive amination reaction, N-terminal modification, thiol Michael addition reaction, disulfide formation reaction, copper(I)-catalyzed alkyne-azide cycloaddition (CuAAC) reaction, strain-promoted alkyne-azide cycloaddtion reaction (SPAAC), Strain-promoted alkyne-nitrone cycloaddition (SPANC), inverse electron-demand Diels-Alder (IEDDA) reaction, oxime/hydrazone formation reaction, free-radical polymerization reaction, or a combination thereof.

Moieties that participate in cycloaddition reactions may be utilized as attachment moieties. Exemplary click moieties and their attachment reactions include (wherein R and R′ represent proteins or substances to which the proteins will be attached):

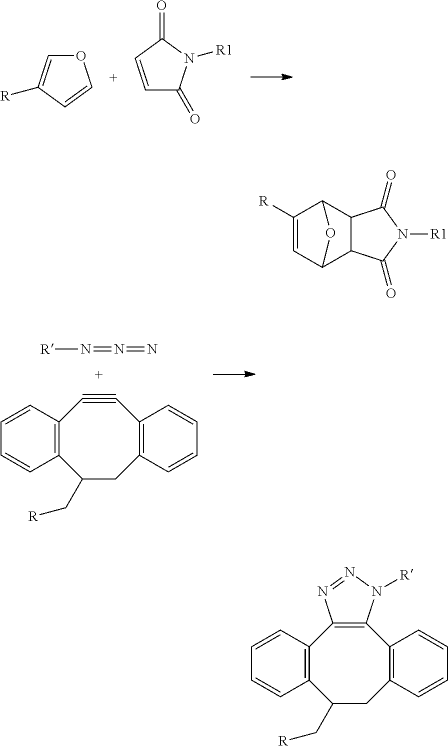

In some cases, moieties that participate in Copper-Catalyzed Azide-Alkyne Cycloadditions (CuAAC) may be utilized as attachment moieties. Optionally, moieties that participate in Strain-Promoted Azide-Alkyne Cycloadditions (SPAAC) may be utilized. For example, an azide moiety can be used as an attachment moiety that is reactive with alkyne moieties. Conversely an alkyne moiety can be used as an attachment moiety that is reactive with azide moieties.

Moieties that participate in inverse-electron demand Diels-Alder (IEDDA) reactions may be utilized as attachment moieties. One of a 1,2,4,5-tetrazine moiety, strained alkene moiety or strained alkyne can be subjected to an IEDDA reaction. Exemplary moieties include, but are not limited to, trans-cyclooctenes, functionalized norbornene derivatives, triazines, or spirohexene. In some cases, a maleimide or furan can be used in a hetero-Diels-Alder cycloaddition between a maleimide and furan. In some cases, a Diels-Alder reaction can achieve covalent coupling of a diene moiety with an alkene moiety to form a six-membered ring complex for attachment, as shown below (wherein R and R1 represent proteins or substances to which the proteins will be attached).

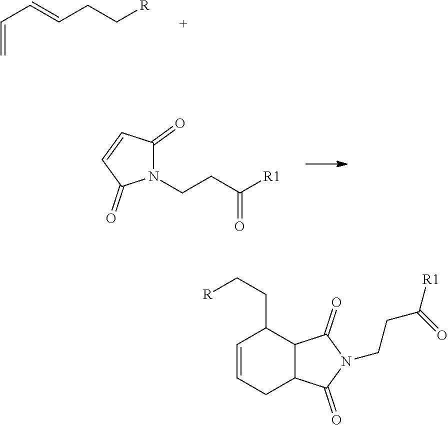

Receptors and ligands can be particularly useful as attachment moieties. A receptor and ligand can bind each other in a non-covalent manner (i.e. forming a non-covalent complex). Optionally, a protein can include a receptor moiety that binds to a ligand moiety on a substance or material of interest, or vice versa. A receptor-ligand pair can be chemically non-reactive and non-catalytic, whereby the chemical structure of the receptor and ligand are not altered upon binding to each other. Exemplary receptors and their ligands include, but are not limited to, an antibody, such as a full-length antibody, or functional fragment thereof, which binds to an epitope; (strept)avidin (or analog thereof) which binds biotin (or analog thereof); complementary nucleic acids which hybridize to each other; nucleic acid aptamers which bind to ligands; lectins which bind carbohydrates; or the like. Affinity reagents and their epitopes, such as those set forth herein below, can also be useful as attachment moieties.

Other useful attachment moieties include components of a SpyTag/SpyCatcher system (See, Zakeri et al. Proceedings Nat'l Acad. Sciences USA. 109 (12): E690-7 (2012)). In this system, a 13 amino acid tag protein (Spy Tag) forms a first coupling handle, with a 12.3 kDa protein (Spy-Catcher) forming the partner to the first coupling handle. The Spy Catcher can irreversibly bond to a Spy Tag to form an attachment. As will be appreciated, either the Spy Tag or Spy Catcher can be attached to an amino acid for use as an attachment moiety. Further attachment moieties are set forth in WO 2019/195633 A1; US Pat. App. Pub. No. 2021/0101930 A1; U.S. Pat. No. 11,203,612; US Pat. App. Pub. No. 2022/0162684 A1; or U.S. patent application Ser. No. 17/692,035, each of which is incorporated herein by reference.

An artificial moiety (e.g. blocking moiety or attachment moiety) can be attached to an amino acid by exploiting the reactivity of side chains of particular amino acids. A precursor of an artificial moiety can include any of a variety of reactive moieties that form covalent bonds with amino acid side chain moieties. Exemplary reactions are set forth below with the amino acids and artificial moieties indicated by R groups (e.g. R or R1). The reactions can also be used to attach the amino acids to other substances or materials (e.g. particles, solid supports, labels, unique identifiers, etc.) having the exemplified reactive moieties.

Primary amines present in lysines or at the amino terminus of a protein can be modified by a variety of reactions. For example, an amine can react with an aldehyde via reductive amination to form an amine attachment. An isothiocyanate can react with an amine to form a thiourea bond.

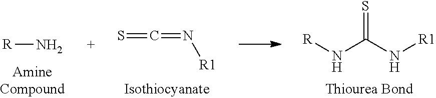

An isocyanate can react with an amine to form an isourea bond.

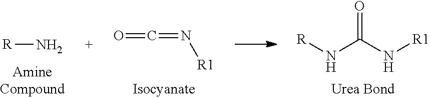

An acyl azide can react with a primary amine to form an amide bond.

An N-hydroxysuccinimide (NHS) ester can react with an amine to form an amide bond.

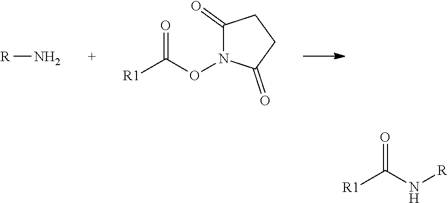

A sulfonyl chloride can react with a primary amine to form a sulfonamide linkage.

An aryl halide, such as fluorobenzene derivative, can form a covalent bond with an amine. Other nucleophiles such as thiol, imidazolyl, and phenolate can also react with an aryl halide to form stable bonds. As shown below, a fluorobenzene can react with an amine to form a substituted aryl amine bond.

An imidoester can react with an amine to form an amidine linkage.

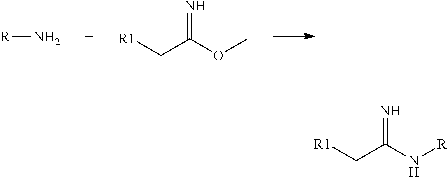

An epoxide or oxirane can react with a nucleophile in a ring-opening process. The reaction can take place with primary amines, sulfhydryls, or hydroxyl groups to create secondary amine, thioether, or ether bonds, respectively.

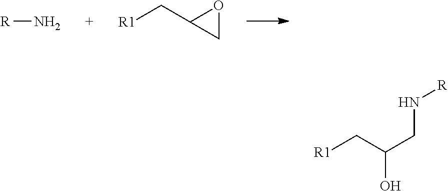

A carbonate can react with a nucleophile to form a carbamate linkage.

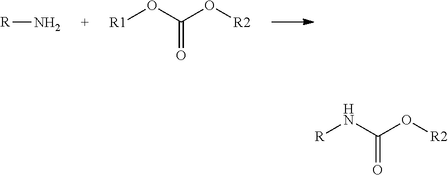

A carbonyl, such as an aldehyde, ketone, or glyoxal, can react with an amine to form a Schiff base intermediate. In some cases, the addition of sodium borohydride or sodium cyanoborohydride will result in reduction of the Schiff base intermediate and covalent bond formation, creating a secondary amine bond.

In some cases, N,N′-Carbonyl diimidazole (CDI) may be used to react with a carboxylate to form N-acylimidazole which can then react with an amine to form an amide bond or with a hydroxyl to form an ester linkage.

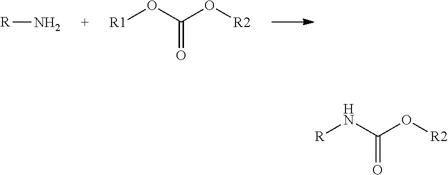

N,N′-disuccinimidyl carbonate moieties and N-hydroxysuccinimidyl chloroformate moieties are reactive toward nucleophiles. These moieties can react with amines or hydroxyls to form stable crosslinked products.

A fluorophenyl ester moiety can react with an amine to form an amide bond. Exemplary fluorophenyl esters include pentafluorophenyl (PFP) ester, tetrafluorophenyl (TFP) ester, or sulfo-tetrafluoro-phenyl (STP) ester.

Sulfhydryls present in cysteines can be modified by a variety of reactions. For example, a maleimide can undergo an alkylation reaction with a sulfhydryl to form a stable thioether bond.

An aziridine can react with nucleophiles. For example, a sulfhydryl can react with an aziridine to form a thioether bond.

An acryloyl can react with a sulfhydryl to form a thioether bond.

An electron-deficient aryl can react with a sulfhydryl to form a substituted aryl bond.

A pyridyl dithiol can undergo an interchange reaction with a free sulfhydryl to yield a mixed disulfide product.

A vinyl sulfone can react with thiols, amines or hydroxyls. The product of the reaction of a thiol with a vinyl sulfone gives a beta-thiosulfonyl linkage.

Carboxyl moieties at the C-terminus of proteins or at amino acid side chains, such as those of glutamate or aspartate, can be modified in a method set forth herein. For example, carbonyldiimidazole (CDI) can be used in non-aqueous conditions to activate carboxylic acids for direct conjugation to primary amines (—NH₂) via amide bonds. Particularly useful carbodiimide compounds include, but are not limited to, 1-ethyl-3-(3-dimethylaminopropyl)carbodiimide (EDC) and dicyclohexylcarbodiimide (DCC).

A method of the present disclosure can include a step of removing post-translational moieties from post-translationally modified amino acids, thereby forming standard amino acids. In some cases, an enzyme can be used to remove a post-translational moiety from an amino acid. An enzyme that removes a post-translational moiety independently of amino acid sequence context surrounding the post-translationally modified amino acid can be used. In other cases, a sequence-specific enzyme can be used to remove a post-translational moiety.

A phosphatase enzyme can be used to remove a phosphate moiety from an amino acid. A broadscale (e.g. sequence agnostic) phosphatase such as alkaline phosphatase can be useful. Protein phosphatases are available for removing phosphate moieties from various types of amino acids. Exemplary protein phosphatases include, but are not limited to, tyrosine-specific kinases such as PTP1B; serine/threonine-specific phosphatases such as PP2C and PPP2CA; dual specificity phosphatases such as lambda protein phosphatase or VHR, both of which can remove phosphate moieties from serine, threonine or tyrosine residues; or histidine phosphatase such as PHP. Phosphatases or kinases that are specific to particular signal transduction pathways can be used to remove phosphates in a sequence specific manner if desired.

Several enzymes are available for removing post-translational moieties from lysines. Examples are set forth in Wang and Cole, Cell Chemical Biology 27:953-969 (2020) (which is incorporated herein by reference) and below. Lysine deacetylases can be used to remove acetyl moieties from lysines. For example, eighteen different protein lysine deacetylases (e.g. histone deacetylases) are known to remove acetyl moieties from lysines in human proteins. Lysine demethylases can be used to remove methyl moieties from lysines. Deubiquitinases (DUBs) are isopeptidases that sever the amide bond between a lysine side chain of a protein and the ubiquitin (Ub) C terminus. Many DUBs can cleave Ub-Ub amide linkages whereas others show selectivity for particular ubiquitinated proteins.

Optionally, glycan moieties can be released from proteins in a method of the present disclosure. For example, N-glycans or O-glycans can be released from glycoproteins using glycosidases. Any of a variety of enzymes can be used to remove glycans from proteins. For example, α2-3,6,8,9-Neuraminidase can be used to cleave non-reducing terminal branched and unbranched sialic acids; β1,4-galactosidase can be used to remove β1,4-linked nonreducing terminal galactose from proteins; β-N-acetylgucosaminidase can be used to cleave non-reducing terminal β-linked N-acetylgucosamine from proteins; endo-a-N-acetylgalactosaminidase can be used to remove O-glycosylation, for example, removing serine- or threonine-linked unsubstituted Galb1,3GalNac; and PNGase F can be used to cleave oligosaccharides from asparagines. Exemplary reagents and methods for releasing glycans from proteins are set forth in Zhang et al. Frontiers in Chemistry, vol 8, Article 508 (2020) doi: 10.3389/fchem.2020.00508, which is incorporated herein by reference.

A method of the present disclosure can include a step of attaching a protein to another substance or material. For example, a step can be carried out to attach a protein to a particle (e.g. structured nucleic acid particle or solid-support particle), label (e.g. luminophore), solid support or unique identifier (e.g. address of an array). Attachment can be achieved using an attachment moiety, such as those set forth above. In some cases, attachment can be achieved using a reactive moiety that is present in the side chain of a standard amino acid. Attachment can also be achieved using a post-translational moiety. As set forth above, attachment can be covalent or non-covalent.

A particle to which a protein will be, or is, attached can be composed of a solid support material, such as those set forth herein. However, a particle need not be composed of solid-support material. A particularly useful particle is a structured nucleic acid particle such as a nucleic acid origami. A nucleic acid origami can include one or more nucleic acids having a variety of overall shapes such as a disk, tile, sphere, cuboid, tubule, pyramid, polyhedron, or combination thereof. Examples of structures formed with DNA origami are set forth in Zhao et al. Nano Lett. 11, 2997-3002 (2011); Rothemund Nature 440:297-302 (2006); Sigle et al, Nature Materials 20:1281-1289 (2021); or U.S. Pat. Nos. 8,501,923 or 9,340,416, each of which is incorporated herein by reference. In some configurations, a nucleic acid origami may include a scaffold nucleic acid and a plurality of staple oligonucleotides. The scaffold can be configured as a single, continuous strand of nucleic acid, and the staples can be formed by nucleic acids that hybridize, in whole or in part, with the scaffold nucleic acid.

A particular advantage of nucleic acid origami is the ability to include a specified number of attachment moieties. For example, a nucleic acid origami can be engineered to include only a single attachment point for a protein or other molecule. However, if desired more than one attachment point can be engineered into a nucleic acid origami. Accordingly, a nucleic acid origami can include at least 1, 2, 3, 4, 5, 10, 15, 20, 25 or more attachment points. Alternatively or additionally, a nucleic acid origami can include at most 25, 20, 15, 10, 5, 4, 3, 2, or 1 attachment point. An attachment point can be in a scaffold nucleic acid or oligonucleotide (e.g. staple) of a nucleic acid origami.

A scaffold nucleic acid can be linear (i.e. having a 3′ end and 5′ end) or circular (i.e. closed such that the scaffold lacks a 3′ end and 5′ end). A scaffold nucleic acid can be single stranded but for a plurality of oligonucleotides hybridized thereto or short regions of internal complementarity. The size of a nucleic acid scaffold may vary to accommodate different uses. For example, a nucleic acid scaffold may include at least about 100, 500, 1000, 2500, 5000 or more nucleotides. Alternatively or additionally, a nucleic acid scaffold may include at most about 5000, 2500, 1000, 500, 100 or fewer nucleotides. Optionally, a scaffold can include an attachment moiety that is configured to form a covalent or non-covalent bond with a reactive moiety, such as an amino acid of a protein.

An oligonucleotide component of an origami can include a first sequence region that is hybridized to a scaffold nucleic acid while a second region of the oligonucleotide is not hybridized to the scaffold. The second region can be in a single stranded state or, alternatively, can participate in a hairpin or other self-annealed structure in the oligonucleotide. Optionally, the second region of the oligonucleotide can include an attachment moiety that is configured to form a covalent or non-covalent bond with a protein moiety, such as a standard amino acid, artificially modified amino acid or post-translationally modified amino acid. Optionally, an oligonucleotide can include two sequence regions that hybridize to a scaffold nucleic acid, for example, to function as a ‘staple’ that restrains the structure of the scaffold. For example, a single oligonucleotide can hybridize to two regions that are separated from each other in the primary sequence of the scaffold. As such, the oligonucleotide can function to retain those two regions of the scaffold in proximity to each other or to otherwise constrain the scaffold to a desired conformation. The oligonucleotides can be linear (i.e. having a 3′ end and a 5′ end) or closed (i.e. circular, lacking both 3′ and 5′ ends). An oligonucleotide that is included in a nucleic acid origami can have a length of at least about 10, 25, 50, 100, 250, 500, or more nucleotides. Alternatively or additionally, an oligonucleotide may have a length of no more than about 500, 250, 100, 50, 25, 10, or fewer nucleotides.

A particle, whether composed of nucleic acid or other material, may have any of a variety of sizes and shapes to accommodate use in a desired application. For example, a particle can have a regular or symmetric shape or, alternatively, a particle can have an irregular or asymmetric shape. The shape can be rigid or pliable. The size or shape of a particle can be characterized with respect to area (e.g. footprint on a surface) or volume. Optionally, a particle can have a minimum, maximum or average area of at least about 100 nm², 1 μm², 10 μm², 100 μm², 1 mm², or more. Alternatively or additionally, a particle can have a minimum, maximum or average area of no more than about 1 mm², 100 μm², 10 μm², 1 μm², 1 nm² or less. Optionally, a particle can have a minimum, maximum or average volume of at least about 1 mm³, 10 mm³, 100 mm³, 1 mm³ or more. Alternatively or additionally, a particle can have a minimum, maximum or average volume of no more than about 1 mm³, 100 mm³, 10 mm³, 1 mm³ or less.

A particle that is made or used in a method set forth herein can be in fluid phase, immobilized on a solid support (i.e. solid phase), or immobilized in another material such as a gel or solid support material. For example, a population of particles can be colloidal for some, or all steps of a method set forth herein. Alternatively, a population of particles can be immobilized in, or on a gel or solid support for some, or all steps of a method set forth herein.

A method of the present disclosure can include a step of attaching a protein to an address of an array. Attachment can be achieved using an attachment moiety, such as those set forth above. In some cases, attachment can be achieved using a reactive moiety that is present in a the side chain of a standard amino acid or post-translationally modified amino acid. As set forth above, attachment can be covalent or non-covalent. In some cases, attachment of a protein to an address of an array can occur via a particle such as a nucleic acid origami particle or other structured nucleic acid particle. For a plurality of proteins, each protein can be located at a discrete address that is resolvable from the addresses for the other proteins in the plurality. For example, each protein can be present at a unique address of an array.

A useful array configuration is a single molecule configuration in which a single protein is attached to each address in the array. For example, individual addresses can each include one, and only one, moiety that is reactive toward a protein. Optionally, addresses of an array can be configured to accommodate one, and only one, particle, and the single particle can be configured to attach one, and only one, protein. As set forth above, nucleic acid origami provides a useful particle that can be configured to attach a single protein. The addresses of an array can have a size and/or shape that accommodates a single particle, such that the presence of the particle precludes other particles from also occupying the address. For example, the address can be a well into which no more than one particle can fit. Similarly, the address can be a pad or post having an area that is smaller than the footprint of the individual particles that are loaded into the addresses. It will be understood that particles need not be used to mediate attachment of proteins to addresses of an array. For example, an array can be manufactured to include addresses that each have one, and only one, attachment moiety.

Particles can be attached to addresses using chemistries set forth herein for attaching proteins to other substances or materials. The chemistries can involve covalent or non-covalent attachment. In either case, the attachment of particles to sold supports (e.g. addresses of an array) can be reversible to facilitate subsequent detachment of the particle from the solid support. For example, cleavable covalent bonds (e.g. chemically cleavable, enzymatically cleavable or photolytically cleavable) can be used or non-covalent bonds (e.g. nucleic acid hybrids, receptor-ligand pairs, ionic interactions, polar interactions, van der Waals forces, hydrogen bonds etc.) can be used. Particles can be detached from solid supports using reagents and methods set forth herein for disrupting crosslinks or using reagents and methods known in the art.

An array of addresses can be arranged in an ordered or repeating pattern such as a line of spatially separated addresses, a curve or spiral of spatially separated addresses, a rectilinear grid of spatially separated addresses, or hexagonal grid of spatially separated addresses. Alternatively, the addresses can be arranged randomly in a non-repeating pattern. For example, addresses need not be present in a repeating pattern on a surface, and instead a lawn of addresses can be randomly located on the surface. Whether addresses are arranged in a repeating or random pattern on a solid support, the spatial separation can be configured such that addresses abut each other or such that addresses are separated by a gap. Accordingly, the average pitch for the addresses of an array can be equivalent to or greater than the longest dimension of the addresses. For example, the average pitch for the addresses can be at least 100 nm, 250 nm, 500 nm, 750 nm, 1 mm, 5 mm, 10 mm or more. Alternatively or additionally, the average pitch for the addresses can be at most 10 mm, 5 mm, 1 mm, 750 nm, 500 nm, 250 nm, 100 nm or less.

A solid support upon which an array is formed can have a planar surface. However, the surface need not be planar. For example, addresses can be configured as contours or features on a solid support such as wells, pits, channels or other concave contours. Optionally, addresses can constitute posts, ridges, or other protruding contours.

A method set forth herein can include a step of attaching a protein to a label. Attachment can be achieved using an attachment moiety, such as those set forth above. In some cases, attachment can be achieved using a reactive moiety that is present in the side chain of a standard amino acid. Attachment can be covalent or non-covalent. In some cases, attachment of a protein to a label can occur via a particle such as a nucleic acid origami particle or other structured nucleic acid particle. Exemplary labels include, but are not limited to, luminophores (e.g. fluorophores), dyes, radioactive isotopes, charge tags and other known signal producing molecules. Labels can be in the form of particles or beads that are encoded with detectable characteristics. For example, particles can be optically encoded with distinguishable luminescence excitation or emission, distinguishable diffraction gratings, or distinguishable images. Size and shape distinctions can also provide encodable characteristics. Labels can be encoded with other distinguishable characteristics such as luminescence lifetime, luminescence polarity, radiofrequency transmission, light absorption wavelength, magnetic properties, and other signal types.

In particular configurations of methods or compositions set forth herein, a label can be a unique identifier label. The composition of the label can include a molecule, bead, particle or other detectable substance such as those set forth herein. A protein can be identified or characterized, for example, using an assay set forth herein, and the resulting identification or characterization can be correlated with an associated unique identifier. A particularly useful unique identifier label is a nucleic acid molecule having a unique nucleotide sequence. A protein can be subjected to an assay, and the resulting identification or characterization can be correlated to a unique sequence that is associated with the unknown protein. Unique sequences of nucleic acids can be readily detected and resolved using known molecular biology techniques such as hybridization of the unique identifier nucleic acid to a complementary nucleic acid probe, sequencing the unique identifier nucleic acid (e.g. using Sanger sequencing or next generation sequencing), detecting the unique identifier nucleic acid using real-time polymerase chain reaction (PCR) or quantitative PCR, or sequence-specific modification of the unique identifier nucleic acid such as via cleavage, insertion, extension or the like.

Optionally, a method of the present disclosure can be configured to attach proteins to a solid support, label or unique identifier via artificially modified amino acids. Accordingly, a method of modifying proteoforms, can include (a) providing a first aliquot from a biological sample and a second aliquot from the biological sample, wherein a plurality of different proteoforms that are present in the first aliquot is also present in the second aliquot, wherein the proteoforms have post-translationally modified amino acids and standard amino acids; (b) treating proteoforms of the first aliquot by: (i) chemically modifying the standard amino acids, thereby forming artificially modified amino acids, (ii) then removing post-translational modifications from the post-translationally modified amino acids, thereby forming standard amino acids, (iii) then attaching the proteoforms of the first aliquot to a label, solid support or unique identifier via artificially modified amino acids formed in step (b)(i); and (c) treating proteoforms of the second aliquot by attaching the proteoforms of the second aliquot to a label, solid support or unique identifier.

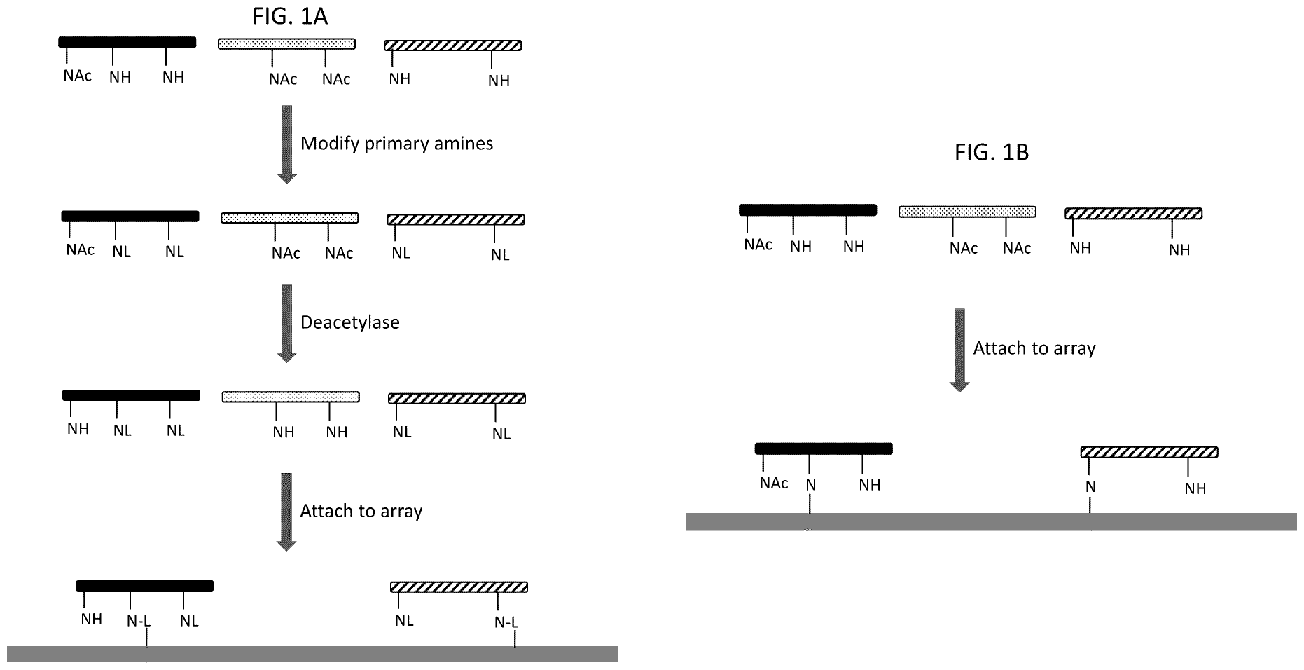

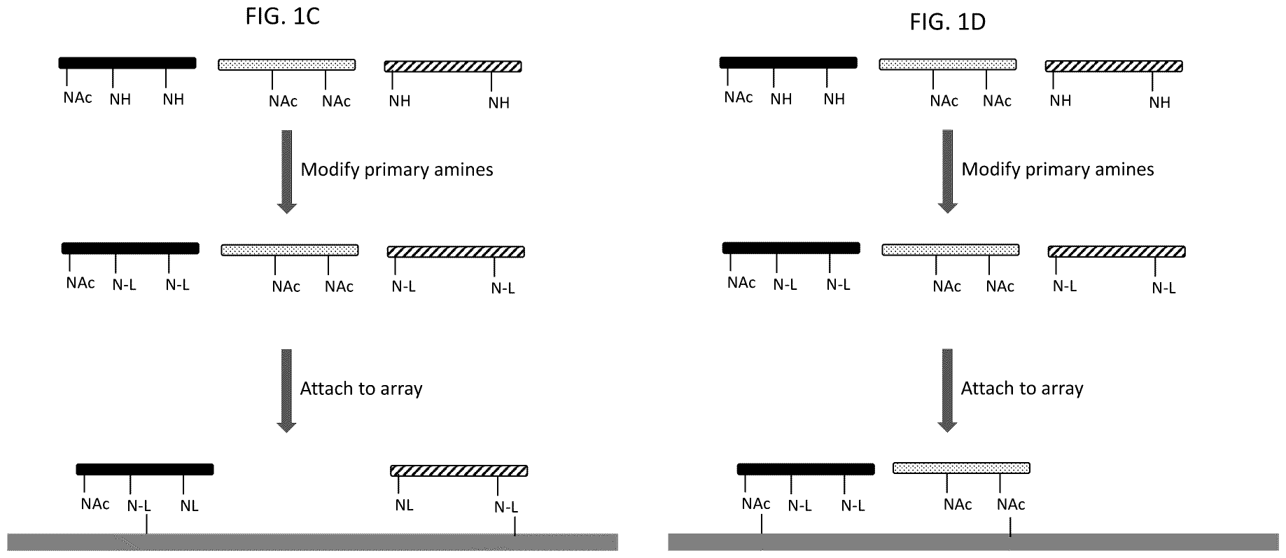

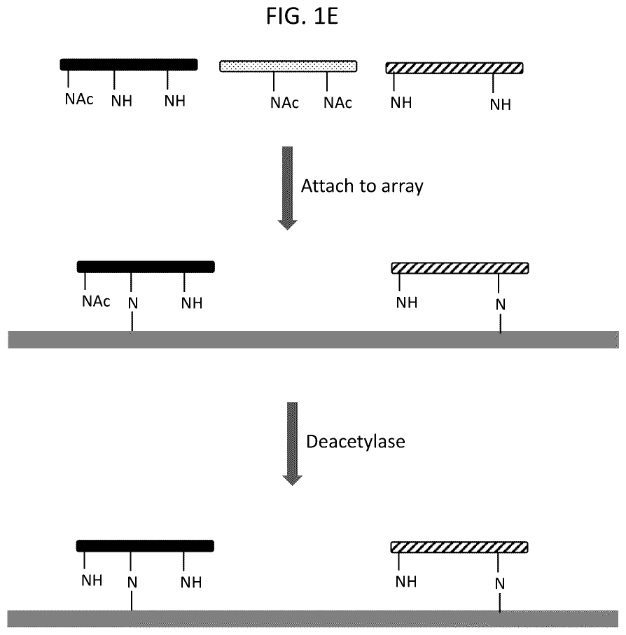

An exemplary method for treating the first aliquot is shown in FIG. 1A in the context of modifying amino moieties of lysine side chains in three different proteoforms. The first proteoform (filled bar) has three amines (N) each being the epsilon amine of a lysine, one of which has an acetyl (Ac) post-translational moiety, the other two being standard lysines (NH). The second proteoform (dotted bar) has two lysines, both of which are acetylated. The third proteoform (hatched bar) has two lysines, both of which are standard lysines. In the first step, the epsilon amines of the proteoforms are modified to add artificial moieties (L). In the second step, a deacetylase enzyme is used to remove the acetyl moieties from the proteoforms. In the third step, the proteoforms are attached to respective addresses of an array via artificial moieties that were added to the proteoforms. In the exemplified configuration, individual addresses each have a single attachment moiety that is reactive with the artificial moieties. As such, each address attaches only a single proteoform and the attachment occurs via only a single amino acid. As a result of the treatment, the first proteoform (filled bar) is stripped of post-translational moietics and is attached to an address via an artificial moiety, the second proteoform (dotted bar) is not attached to an address due to absence of any artificially modified amino acids, and the third proteoform (hatched bar) is attached to an address via an artificial moiety.

A method of the present disclosure can be configured to provide two protein aliquots from a sample, differentially modify proteins of the first aliquot compared to the second aliquot, attach proteins from the first aliquot to a solid support, label or unique identifier via artificially modified amino acids, and attach proteins from the second aliquot to a label, solid support or unique identifier via standard amino acids. Accordingly, a method of modifying proteoforms, can include (a) providing a first aliquot from a biological sample and a second aliquot from the biological sample, wherein a plurality of different proteoforms that are present in the first aliquot is also present in the second aliquot, wherein the proteoforms have post-translationally modified amino acids and standard amino acids; (b) treating proteoforms of the first aliquot by: (i) chemically modifying the standard amino acids, thereby forming artificially modified amino acids, (ii) then removing post-translational modifications from the post-translationally modified amino acids, thereby forming standard amino acids, (iii) then attaching the proteoforms of the first aliquot to a label, solid support or unique identifier via artificially modified amino acids formed in step (b)(i); and (c) treating proteoforms of the second aliquot by attaching the proteoforms of the second aliquot to a label, solid support or unique identifier via standard amino acids.

An exemplary method for treating the first aliquot in the above method is shown in FIG. 1A, as set forth above. The second aliquot can be treated as exemplified in FIG. 1B and as follows. The same proteoforms are present in both aliquots and are represented as set forth above in the context of FIG. 1A. As shown in FIG. 1B, the epsilon amines of the proteoforms are attached to respective addresses of an array. In the exemplified configuration, individual addresses each have a single attachment moiety that is reactive with the artificial moieties. As such, each address attaches only a single proteoform and the attachment occurs via only a single amino acid. As a result of the treatment shown in FIG. 1B, the first proteoform (filled bar) maintains its post-translational moiety and is attached to an address via a standard lysine, the second proteoform (dotted bar) is not attached to an address due to absence of any standard lysines, and the third proteoform (hatched bar) is attached to an address via a standard lysine. The example of FIG. 1B can be modified to attach the proteoforms to respective addresses via post-translational moieties instead of via standard amino acids. As a result, the first proteoform (filled bar) would be attached to an address via the post-translationally modified lysine, the second proteoform (dotted bar) would be attached to an address via one of the post-translationally modified lysines, and the third proteoform (hatched bar) would not be attached to an address due to absence of any post-translational moieties.

A method of the present disclosure can be configured to provide two protein aliquots from a sample, differentially modify proteins of the first aliquot compared to the second aliquot, attach proteins from the first aliquot to a solid support, label or unique identifier via artificially modified amino acids, and attach proteins from the second aliquot to a label or solid support via artificially modified amino acids. Accordingly, a method of modifying proteoforms, can include (a) providing a first aliquot from a biological sample and a second aliquot from the biological sample, wherein a plurality of different proteoforms that are present in the first aliquot is also present in the second aliquot, wherein the proteoforms have post-translationally modified amino acids and standard amino acids; (b) treating proteoforms of the first aliquot by: (i) chemically modifying the standard amino acids, thereby forming artificially modified amino acids, (ii) then removing post-translational modifications from the post-translationally modified amino acids, thereby forming standard amino acids, (iii) then attaching the proteoforms of the first aliquot to a label, solid support or unique identifier via artificially modified amino acids formed in step (b)(i); and (c) treating proteoforms of the second aliquot by: (i) chemically modifying the standard amino acids, thereby forming artificially modified amino acids, and (ii) then attaching the proteoforms of the second aliquot to a label, solid support or unique identifier via artificially modified amino acids formed in step (c)(i).

An exemplary method for treating the first aliquot in the above method is shown in FIG. 1A, as set forth above. The second aliquot can be treated as exemplified in FIG. 1C and as follows. The same proteoforms are present in both aliquots and are represented as set forth above in the context of FIG. 1A. As shown in FIG. 1C, the epsilon amines of the proteoforms are modified in a first step to add artificial moieties (L). In the second step, the proteoforms are attached to respective addresses of an array via artificial moieties that were added to the proteoforms. In the exemplified configuration, individual addresses each have a single attachment moiety that is reactive with the artificial moieties. As such, each address attaches only a single proteoform and the attachment occurs via only a single amino acid. As a result of the treatment shown in FIG. 1C, the first proteoform (filled bar) maintains its post-translational moiety and is attached to an address via an artificial moiety, the second proteoform (dotted bar) is not attached to an address due to absence of any standard lysines, and the third proteoform (hatched bar) is attached to an address via an artificial moiety.

A method of the present disclosure can be configured to provide two protein aliquots from a sample, differentially modify proteins of the first aliquot compared to the second aliquot, attach proteins from the first aliquot to a solid support, label or unique identifier via artificially modified amino acids, and attach proteins from the second aliquot to a label, solid support or unique identifier via post-translationally modified amino acids. Accordingly, a method of modifying proteoforms, can include (a) providing a first aliquot from a biological sample and a second aliquot from the biological sample, wherein a plurality of different proteoforms that are present in the first aliquot is also present in the second aliquot, wherein the proteoforms have post-translationally modified amino acids and standard amino acids; (b) treating proteoforms of the first aliquot by: (i) chemically modifying the standard amino acids, thereby forming artificially modified amino acids, (ii) then removing post-translational modifications from the post-translationally modified amino acids, thereby forming standard amino acids, (iii) then attaching the proteoforms of the first aliquot to a label, solid support or unique identifier via artificially modified amino acids formed in step (b)(i); and (c) treating proteoforms of the second aliquot by: (i) chemically modifying the standard amino acids, thereby forming artificially modified amino acids, and (ii) then attaching the proteoforms of the second aliquot to a label, solid support or unique identifier via post-translationally modified amino acids.

An exemplary method for treating the first aliquot in the above method is shown in FIG. 1A, as set forth above. The second aliquot can be treated as exemplified in FIG. 1D and as follows. The same proteoforms are present in both aliquots and are represented as set forth above in the context of FIG. 1A. As shown in FIG. 1D, the epsilon amines of the proteoforms are modified in a first step to add artificial moieties (L). In the second step, the proteoforms are attached to respective addresses of an array via post-translational moieties. In the exemplified configuration, individual addresses each have a single attachment moiety that is reactive with the artificial moieties. As such, each address attaches only a single proteoform and the attachment occurs via only a single amino acid. As a result of the treatment shown in FIG. 1D, the first proteoform (filled bar) is attached to an address via a post-translational moiety, the second proteoform (dotted bar) is attached to an address via the post-translational moiety, and the third proteoform (hatched bar) is not attached to an address due to absence of post-translational moieties. It will be understood that the first step of FIG. 1D need not be carried out. In this case, the same post-translational moieties are available for attaching the proteoforms to addresses; however, the attached proteoforms will lack artificial moieties.

The methods exemplified in FIGS. 1A to 1D demonstrate modification of proteoforms prior to being attached to an array. In some cases, it can be useful to modify proteoforms after attachment to an array. As exemplified by FIG. 1E, proteoforms in a first aliquot can be attached to an address of an array via a standard version of an amino acid (e.g. lysine) and then the attached proteoform can be treated to remove post-translational moieties (e.g. acetyl) from post-translationally modified versions of the amino acid. As a result of the treatment shown in FIG. 1E, the first proteoform (filled bar) is stripped of its post-translational moiety and is attached to an address via a standard lysine, the second proteoform (dotted bar) is not attached to an address due to absence of any standard lysines, and the third proteoform (hatched bar) is attached to an address via a standard lysine.

It will be understood that the methods exemplified in the context of FIGS. 1A to 1E need not be limited to evaluating lysines, nor to lysines having acetyl modifications. Rather the methods can be extended to other amino acid types and other post-translational modifications. Moreover, the methods can be extended to include attachment to substances or materials other than arrays. For example, the attachment steps can instead use particles, labels or solid supports that do not necessarily have arrays of addresses.

Optionally, a method of the present disclosure can be configured to attach proteins to a solid support, particle, label or unique identifier via standard amino acids. Accordingly, a method of modifying proteoforms, can include (a) providing a first aliquot from a biological sample and a second aliquot from the biological sample, wherein a plurality of different proteoforms that are present in the first aliquot is also present in the second aliquot, wherein the proteoforms have post-translationally modified amino acids and standard amino acids; (b) treating proteoforms of the first aliquot by: (i) chemically modifying the standard amino acids, thereby forming artificially modified amino acids, (ii) then removing post-translational modifications from the post-translationally modified amino acids, thereby forming standard amino acids, (iii) then attaching the proteoforms of the first aliquot to a label, particle, solid support or unique identifier via standard amino acids formed in step (b)(ii); and (c) treating proteoforms of the second aliquot by attaching the proteoforms of the second aliquot to a label, particle, solid support or unique identifier.

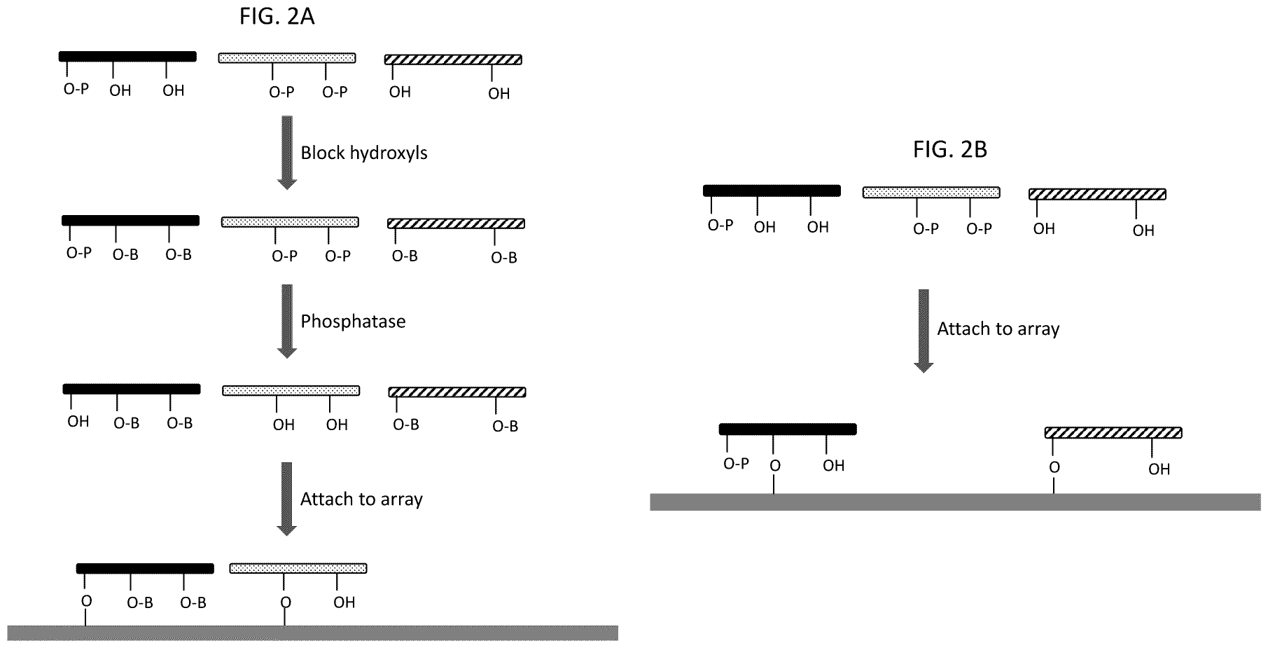

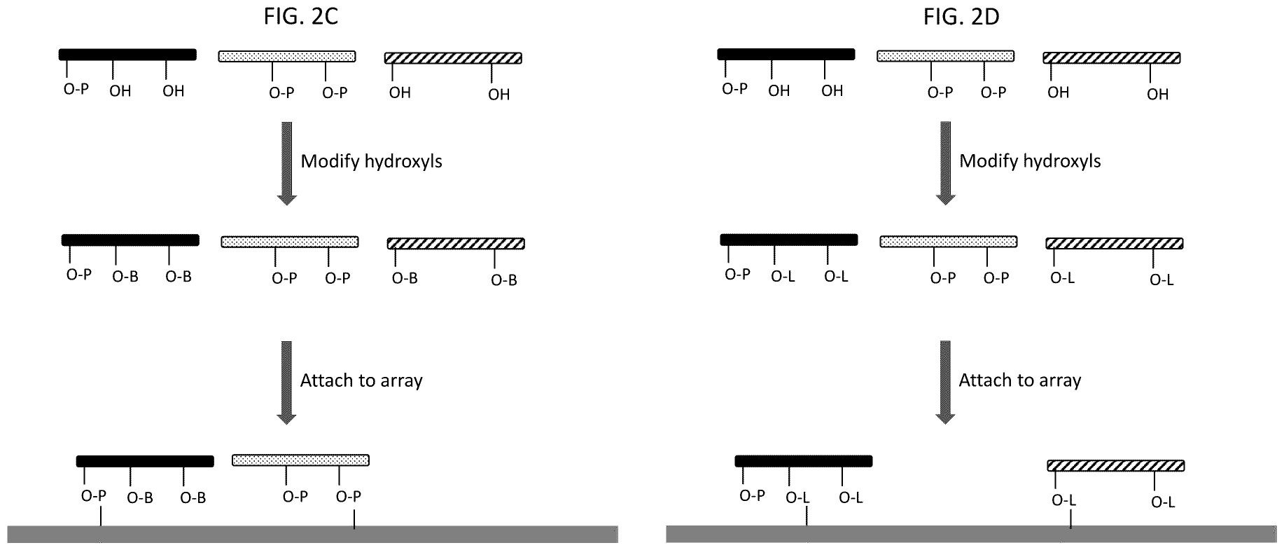

An exemplary method for treating the first aliquot in the above method is shown in FIG. 2A in the context of modifying hydroxyl moieties of serine side chains in three different proteoforms. The first proteoform (filled bar) has three oxygens (O) each being in the side chain of a serine, one of which has a phosphoryl (P) post-translational moiety, the other two being standard serines (OH). The second proteoform (dotted bar) has two serines, both of which are phosphorylated. The third proteoform (hatched bar) has two serines, both of which are standard serines. In the first step, the hydroxyls of standard serines in the proteoforms are modified to add blocking moieties (B). The blocking moieties are inert to subsequent steps shown in FIG. 2A. In the second step, a phosphatase enzyme is used to remove the phosphoryl moieties from the proteoforms. In the third step, the proteoforms are attached to respective addresses of an array via oxygens of dephosphorylated serines. In the exemplified configuration, individual addresses each have a single attachment moiety that is reactive with the serine oxygens. As such, each address attaches only a single proteoform and the attachment occurs via only a single amino acid. As a result of the treatment, the first proteoform (filled bar) is stripped of post-translational moieties and is attached to an address via a standard serine, the second proteoform (dotted bar) is attached to an address via a standard serine, and the third proteoform (hatched bar) is not attached to an address due to absence of post-translational moieties.

A method of the present disclosure can be configured to provide two protein aliquots from a sample, differentially modify proteins of the first aliquot compared to the second aliquot, attach proteins from the first aliquot to a solid support, particle, label or unique identifier via standard amino acids, and attach proteins from the second aliquot to a label, particle, solid support or unique identifier via standard amino acids. Accordingly, a method of modifying proteoforms, can include (a) providing a first aliquot from a biological sample and a second aliquot from the biological sample, wherein a plurality of different proteoforms that are present in the first aliquot is also present in the second aliquot, wherein the proteoforms have post-translationally modified amino acids and standard amino acids; (b) treating proteoforms of the first aliquot by: (i) chemically modifying the standard amino acids, thereby forming artificially modified amino acids, (ii) then removing post-translational modifications from the post-translationally modified amino acids, thereby forming standard amino acids, (iii) then attaching the proteoforms of the first aliquot to a label, particle, solid support or unique identifier via standard amino acids formed in step (b)(ii); and (c) treating proteoforms of the second aliquot by attaching the proteoforms of the second aliquot to a label, particle, solid support or unique identifier via standard amino acids.

An exemplary method for treating the first aliquot in the above method is shown in FIG. 2A, as set forth above. The second aliquot can be treated as exemplified in FIG. 2B and as follows. The same proteoforms are present in both aliquots and are represented as set forth above in the context of FIG. 2A. As shown in FIG. 2B, the proteoforms are attached to respective addresses of an array via oxygens of standard serines. In the exemplified configuration, individual addresses each have a single attachment moiety that is reactive with the serines. As such, each address attaches only a single proteoform and the attachment occurs via only a single amino acid. As a result of the treatment shown in FIG. 2B, the first proteoform (filled bar) maintains its post-translational moiety and is attached to an address via a serine oxygen, the second proteoform (dotted bar) is not attached to an address due to absence of any standard serines, and the third proteoform (hatched bar) is attached to an address via a serine oxygen. The example of FIG. 2B can be modified to attach the proteoforms to respective addresses via post-translational moieties instead of via standard amino acids. As a result, the first proteoform (filled bar) would be attached to an address via the post-translationally modified serine, the second proteoform (dotted bar) would be attached to an address via one of the post-translationally modified serines, and the third proteoform (hatched bar) would not be attached to an address due to absence of any post-translational moieties.

A method of the present disclosure can be configured to provide two protein aliquots from a sample, differentially modify proteins of the first aliquot compared to the second aliquot, attach proteins from the first aliquot to a solid support, particle, label or unique identifier via standard amino acids, and attach proteins from the second aliquot to a label, particle, solid support or unique identifier via post-translationally modified amino acids. Accordingly, a method of modifying proteoforms, can include (a) providing a first aliquot from a biological sample and a second aliquot from the biological sample, wherein a plurality of different proteoforms that are present in the first aliquot is also present in the second aliquot, wherein the proteoforms have post-translationally modified amino acids and standard amino acids; (b) treating proteoforms of the first aliquot by: (i) chemically modifying the standard amino acids, thereby forming artificially modified amino acids, (ii) then removing post-translational modifications from the post-translationally modified amino acids, thereby forming standard amino acids, (iii) then attaching the proteoforms of the first aliquot to a label, particle, solid support or unique identifier via standard amino acids formed in step (b)(ii); and (c) treating proteoforms of the second aliquot by: (i) chemically modifying the standard amino acids, thereby forming artificially modified amino acids, and (ii) then attaching the proteoforms of the second aliquot to a label, particle, solid support or unique identifier via post-translationally modified amino acids.

An exemplary method for treating the first aliquot in the above method is shown in FIG. 2A, as set forth above. The second aliquot can be treated as exemplified in FIG. 2C and as follows. The same proteoforms are present in both aliquots and are represented as set forth above in the context of FIG. 2A. In the first step of FIG. 2C, the hydroxyls of standard serines in the proteoforms are modified to add blocking moieties (B). In the second step, the proteoforms are attached to respective addresses of an array via phosphoryl moieties of post-translationally modified serines. For the method of FIG. 2C, the first step in is optional and need not be performed, for example, in situations where free hydroxyls are not expected to react in the second step. In the exemplified configuration, individual addresses each have a single attachment moiety that is reactive with the phosphates. As such, each address attaches only a single proteoform and the attachment occurs via only a single amino acid. As a result of the treatment shown in FIG. 2C, the first proteoform (filled bar) is attached to an address via a post-translationally modified serine, the second proteoform (dotted bar) is attached to an address via a post-translationally modified serine, and the third proteoform (hatched bar) is not attached to an address due to absence of any phosphoryl moieties.

A method of the present disclosure can be configured to provide two protein aliquots from a sample, differentially modify proteins of the first aliquot compared to the second aliquot, attach proteins from the first aliquot to a solid support, particle, label or unique identifier via standard amino acids, and attach proteins from the second aliquot to a label, particle, solid support or unique identifier via artificially modified amino acids. Accordingly, a method of modifying proteoforms, can include (a) providing a first aliquot from a biological sample and a second aliquot from the biological sample, wherein a plurality of different proteoforms that are present in the first aliquot is also present in the second aliquot, wherein the proteoforms have post-translationally modified amino acids and standard amino acids; (b) treating proteoforms of the first aliquot by: (i) chemically modifying the standard amino acids, thereby forming artificially modified amino acids, (ii) then removing post-translational modifications from the post-translationally modified amino acids, thereby forming standard amino acids, (iii) then attaching the proteoforms of the first aliquot to a label, particle, solid support or unique identifier via standard amino acids formed in step (b)(ii); and (c) treating proteoforms of the second aliquot by: (i) chemically modifying the standard amino acids, thereby forming artificially modified amino acids, and (ii) then attaching the proteoforms of the second aliquot to a label, particle, solid support or unique identifier via artificially modified amino acids.

An exemplary method for treating the first aliquot in the above method is shown in FIG. 2A, as set forth above. The second aliquot can be treated as exemplified in FIG. 2D and as follows. The same proteoforms are present in both aliquots and are represented as set forth above in the context of FIG. 2A. In the first step of FIG. 2D, the hydroxyls of standard serines in the proteoforms are modified to add artificial moieties (L). In the second step, the proteoforms are attached to respective addresses of an array via artificial moieties. In the exemplified configuration, individual addresses each have a single attachment moiety that is reactive with the artificial moieties. As such, each address attaches only a single proteoform and the attachment occurs via only a single amino acid. As a result of the treatment shown in FIG. 2D, the first proteoform (filled bar) retains the post-translational moiety and is attached to an address via an artificially modified serine, the second proteoform (dotted bar) is not attached to an address due to absence of any artificial moieties, and the third proteoform (hatched bar) is attached to an address via an artificial moiety.

It will be understood that the methods exemplified in the context of FIGS. 2A to 2D need not be limited to evaluating serines, nor to serines having phosphoryl modifications. Rather the methods can be extended to other amino acid types and other post-translational modifications. Moreover, the methods can be extended to include attachment to substances or materials other than arrays. For example, the attachment steps can instead use particles, labels or solid supports that do not necessarily have arrays of addresses.

An amino acid can be artificially modified to alter reactivity toward a label, particle, solid support, or unique identifier in comparison to the standard version of the amino acid and/or in comparison to a post-translationally modified version of the amino acid. For example, an artificial moiety of an artificially modified version of an amino acid can function to prevent or inhibit a reaction that is capable of occurring for the standard version of the amino acid or for a post-translationally modified version of the amino acid. For example, the artificial moiety can be a blocking moiety that removes or alters a reactive moiety of the standard amino acid. Alternatively, an artificial moiety of an artificially modified version of an amino acid can provide reactivity that is not characteristic for the standard version of the amino acid or for a post-translationally modified version of the amino acid. For example, the artificial moiety can be an attachment moiety or reactive moiety that is not present in the standard amino acid.

An artificially modified version of an amino acid can have orthogonal reactivity in comparison to a standard version of the amino acid or to a post-translationally modified version of the amino acid. For example, an artificially modified version of an amino acid can have increased reactivity with a label, particle, solid support, or unique identifier in comparison to reactivity of a standard version of the amino acid and/or to a post-translationally modified version of the amino acid. Alternatively, an artificially modified version of an amino acid can have decreased reactivity with a label, particle, solid support, or unique identifier in comparison to reactivity of a standard version of the amino acid and/or a post-translationally modified version of the amino acid.

A standard version of an amino acid can have orthogonal reactivity in comparison to an artificially modified version of the amino acid or to a post-translationally modified version of the amino acid. For example, a standard version of an amino acid can have increased reactivity with a label, particle, solid support, or unique identifier in comparison to reactivity of an artificially modified version of the amino acid and/or to a post-translationally modified version of the amino acid. Alternatively, a standard version of an amino acid can have decreased reactivity with a label, particle, solid support, or unique identifier in comparison to reactivity of an artificially modified version of the amino acid and/or a post-translationally modified version of the amino acid.

A post-translationally modified version of an amino acid can have orthogonal reactivity in comparison to a standard version of the amino acid or to a post-translationally modified version of the amino acid. For example, a post-translationally modified version of an amino acid can have increased reactivity with a label, particle, solid support, or unique identifier in comparison to reactivity of a standard version of the amino acid and/or to a post-translationally modified version of the amino acid. Alternatively, a post-translationally modified version of an amino acid can have decreased reactivity with a label, particle, solid support, or unique identifier in comparison to reactivity of a standard version of the amino acid and/or a post-translationally modified version of the amino acid.

### Differential Detection of Proteoforms

A method of the present disclosure can be configured to detect proteins such as proteoforms. For example, a method set forth herein for modifying proteoforms can include a step of detecting proteoforms. Accordingly, the present disclosure provides a method of detecting proteoforms, including (a) providing a first aliquot from a biological sample and a second aliquot from the biological sample, wherein a plurality of different proteoforms that are present in the first aliquot is also present in the second aliquot, wherein the proteoforms have post-translationally modified amino acids and standard amino acids; (b) treating proteoforms of the first aliquot by: (i) chemically modifying the standard amino acids, thereby forming artificially modified amino acids, (ii) then removing post-translational modifications from the post-translationally modified amino acids, thereby forming standard amino acids, (iii) then attaching the proteoforms of the first aliquot to a label, particle, solid support, or unique identifier, and (iv) detecting proteoforms of the first aliquot; (c) treating proteoforms of the second aliquot by (i) attaching the proteoforms of the second aliquot to a label, particle, solid support, or unique identifier, and (ii) detecting proteoforms of the second aliquot.

The present disclosure further provides a method of detecting proteoforms, including steps of: (a) providing a first aliquot from a biological sample and a second aliquot from the biological sample, wherein a plurality of different proteoforms that are present in the first aliquot is also present in the second aliquot, wherein the proteoforms have post-translationally modified amino acids and standard amino acids; (b) treating proteoforms of the first aliquot by: (i) chemically modifying the standard amino acids, thereby forming artificially modified amino acids, (ii) then removing post-translational modifications from the post-translationally modified amino acids, thereby forming standard amino acids, and (iii) then detecting proteoforms of the first aliquot; (c) detecting proteoforms of the second aliquot; and (d) identifying presence of a post-translational modification in a proteoform of the biological sample based on differential detection of a proteoform in the first aliquot and in the second aliquot.

Proteoforms can be detected by detecting standard amino acids, post-translationally modified amino acids and/or artificially modified amino acids. A proteoform that has been differentially modified in two or more different aliquots can be detected, and differences in detectability of the differentially modified proteoforms can be analyzed to determine the presence, absence or location of post-translationally modified amino acids and standard amino acids. Any of a variety of methods can be used to detect an amino acid position of a proteoform in a way that distinguishes whether it is the standard version of the amino acid, a post-translationally modified version of the amino acid or an artificially modified version of the amino acid. In some cases, merely distinguishing the standard version of an amino acid from a modified version of the amino acid provides useful information. For example, such information can be correlated with a given phenotype of a sample from which a proteoform is derived, such as state of health, diagnosis of a disease or condition, or prognosis of a disease or condition. In other cases, the identity of the type of post-translational modification that is present at a particular amino acid position can be determined. The added detail regarding the modification can be further useful for a more specific evaluation of a sample, for example, regarding state of health, diagnosis of a disease or condition, or prognosis of a disease or condition.

A proteoform can be characterized based on differential treatment of the proteoform in two separate aliquots from a biological sample and detection of differences between the aliquots. A copy of the proteoform in the first aliquot can be treated to add artificial moieties to standard versions of a particular amino acid, remove post-translational moieties from post-translationally modified versions of the amino acid, and attach the proteoform to an address of an array via an artificial moiety on the modified proteoform. See, for example, FIG. 1A, wherein the amino acid is lysine, the post-translational moiety is acetyl (Ac) and the artificial moiety is indicated as “L”. A copy of the proteoform in the second aliquot can be treated to attach the proteoform to an address of an array via the standard version of a lysine. See, for example, FIG. 1B, wherein the amino acid is lysine and the post-translational moiety is acetyl (Ac). Comparison of the array of FIG. 1A to the array of FIG. 1B, shows that the proteoform represented by the black bar is differentially modified having a standard version of a lysine in the first aliquot and having a post-translationally modified version of the lysine in the second aliquot, thereby indicating the presence of the post-translational modification in the biological sample. The comparison also shows that (1) the proteoform represented by the dotted bar does not attach to the array in either aliquot, thereby indicating that the proteoform does not have any standard lysines in the biological sample; and (2) the proteoform indicated by the hatched bar attaches to an address in both aliquots and lacks post-translational moieties in both aliquots, thereby indicating that the proteoform does not have any post-translationally modified lysines in the biological sample.

Another example is demonstrated by comparison of FIG. 1A to FIG. 1C. A first aliquot can be treated as set forth above and in FIG. 1A. A copy of the proteoform in the second aliquot can be treated to add artificial moieties to standard versions of a particular amino acid, and to attach the proteoform to an address of an array via an artificial moiety on the modified proteoform. See, for example, FIG. 1C, wherein the amino acid is lysine, the post-translational moiety is acetyl (Ac) and the artificial moiety is indicated as “L”. Comparison of the array of FIG. 1A to the array of FIG. 1C, shows that the proteoform represented by the black bar is differentially modified having a standard version of a lysine in the first aliquot and having a post-translationally modified version of the lysine in the second aliquot, thereby indicating the presence of the post-translational modification in the biological sample. The comparison also shows that (1) the proteoform represented by the dotted bar does not attach to the array in either aliquot, thereby indicating that the proteoform does not have any standard lysines; and (2) the proteoform indicated by the hatched bar does not have any post-translationally modified lysines in either aliquot, thereby indicating that the proteoform is not post-translationally modified in the biological sample.

Comparison of FIG. 1A to FIG. 1D provides another example. A first aliquot can be treated as set forth above and in FIG. 1A. A copy of the proteoform in the second aliquot can be treated to add artificial moieties to standard versions of a particular amino acid and attach the proteoform to an address of an array via a post-translational moiety on the modified proteoform. See, for example, FIG. 1D, wherein the amino acid is lysine, the post-translational moiety is acetyl (Ac) and the artificial moiety is indicated as “L”. Comparison of the array of FIG. 1A to the array of FIG. 1D, shows that the proteoform represented by the black bar is differentially attached, being attached via an artificial moiety of a lysine in the first aliquot and being attached via a post-translationally modified lysine in the second aliquot, thereby indicating the presence of the post-translational modification in the biological sample. The comparison also shows that (1) the proteoform represented by the dotted bar attaches to the array in the second aliquot but not the first aliquot, thereby indicating that the proteoform does not have any standard lysines in the biological sample; and (2) the proteoform indicated by the hatched bar attaches to the array in the first aliquot but not the second aliquot, thereby indicating that the proteoform does not have any post-translationally modified lysines in the biological sample.

Proteoforms can be modified prior to being attached to an array, for example, as demonstrated by the examples of FIGS. 1A to 1D. In some cases, it can be useful to modify proteoforms after attachment to an array. For example, a copy of a proteoform in a first aliquot can be attached to an address of an array via a standard version of an amino acid and then the attached proteoform can be treated to remove post-translational moieties from post-translationally modified versions of the amino acid. See, for example, FIG. 1E, wherein the amino acid is lysine and the post-translational moiety is acetyl (Ac). Treatment of a copy of the proteoform in a second aliquot can be carried out as shown in FIG. 1B and comparison of the resulting array with the array of FIG. 1E shows that the proteoform represented by the black bar is differentially modified having a standard version of a lysine in the first aliquot and having a post-translationally modified version of the lysine in the second aliquot, thereby indicating the presence of the post-translational modification in the biological sample. The comparison also shows that (1) the proteoform represented by the dotted bar does not attach to the array in either aliquot, thereby indicating that the proteoform does not have any standard lysines; and (2) the proteoform indicated by the hatched bar attaches to an address in both aliquots and lacks post-translational moieties in both aliquots, thereby indicating that the proteoform does not have any post-translationally modified lysines in the biological sample.

Comparison of the array of FIG. 1E to the array of FIG. 1C, shows that the proteoform represented by the black bar is differentially modified having a standard version of a lysine in the first aliquot (FIG. 1E) and having a post-translationally modified version of the lysine in the second aliquot (FIG. 1C), thereby indicating the presence of the post-translational modification in the biological sample. The comparison also shows that (1) the proteoform represented by the dotted bar does not attach to the array in either aliquot, thereby indicating that the proteoform does not have any standard lysines; and (2) the proteoform indicated by the hatched bar attaches to an address in both aliquots and lacks post-translational moieties in both aliquots, thereby indicating that the proteoform does not have any post-translationally modified lysines in the biological sample.

Comparison of FIG. 1E to FIG. 1D shows that the proteoform represented by the black bar is differentially attached, being attached via a standard lysine in the first aliquot (FIG. 1E) and being attached via a post-translationally modified lysine in the second aliquot (FIG. 1D), thereby indicating the presence of the post-translational modification in the biological sample. The comparison also shows that (1) the proteoform represented by the dotted bar attaches to the array in the second aliquot but not the first aliquot, thereby indicating that the proteoform does not have any standard lysines in the biological sample; and (2) the proteoform indicated by the hatched bar attaches to the array in the first aliquot but not the second aliquot, thereby indicating that the proteoform does not have any post-translationally modified lysines in the biological sample.

Further examples of characterizing proteoforms based on differential modifications are demonstrated by FIGS. 2A to 2C. A copy of a proteoform in a first aliquot can be treated to add blocking moieties to standard versions of a particular amino acid, remove post-translational moieties from post-translationally modified versions of the amino acid, and attach the proteoform to an address of an array via a standard version of the amino acid. Sec, for example, FIG. 2A, wherein the amino acid is serine, the post-translational moiety is phosphate (P) and the blocking moiety is indicated as “B”. A copy of the proteoform in a second aliquot can be treated to attach the proteoform to an address of an array via the standard version of an amino acid that was modified in the first aliquot. See, for example, FIG. 2B, wherein the amino acid is serine and the post-translational moiety is phosphate (P). Comparison of the array of FIG. 2A to the array of FIG. 2B, shows that the proteoform represented by the black bar is differentially modified having a standard version of a serine in the first aliquot and having a post-translationally modified version of the serine in the second aliquot thereby indicating the presence of the post-translational modification in the biological sample. The comparison also shows that (1) the proteoform represented by the dotted bar attaches to the array in the first aliquot but not the second aliquot, thereby indicating that the proteoform does not have any standard serines; and (2) the proteoform indicated by the hatched bar attaches to the array in the second aliquot but not the first aliquot, thereby indicating that the proteoform does not have any post-translationally modified serines.

Another example is demonstrated by comparison of FIG. 2A to FIG. 2C. A first aliquot can be treated as set forth above and in FIG. 2A. A copy of the proteoform in the second aliquot can be treated to add blocking moieties to standard versions of a particular amino acid, and to attach the proteoform to an address of an array via a post-translational moiety on the modified proteoform. Sec, for example, FIG. 2C, wherein the amino acid is serine, the post-translational moiety is phosphate (P) and the blocking moiety is indicated as “B”. Comparison of the array of FIG. 2A to the array of FIG. 2C, shows that the proteoform represented by the black bar is differentially attached to addresses via a standard version of serine and a post-translationally modified serine, respectively, thereby indicating the presence of the post-translational modification in the biological sample. The comparison also shows that the proteoform represented by the dotted bar is differentially attached via a standard version of serine and a post-translationally modified serine, respectively, and is also differentially modified with a post-translational moiety, thereby indicating that the proteoform does not have any standard serines. The proteoform indicated by the hatched bar attaches to an address in the second aliquot but not in the first aliquot, thereby indicating that the proteoform does not have any post-translationally modified serines in the biological sample.

Yet another example is demonstrated by comparison of FIG. 2A to FIG. 2D. A first aliquot can be treated as set forth above and in FIG. 2A. A copy of the proteoform in the second aliquot can be treated to add artificial moieties to standard versions of a particular amino acid, and to attach the proteoform to an address of an array via an artificial moiety on the modified proteoform. Sec, for example, FIG. 2D, wherein the amino acid is serine, the post-translational moiety is phosphate (P) and the artificial moiety is indicated as “L”. Comparison of the array of FIG. 2A to the array of FIG. 2D, shows that the proteoform represented by the black bar is differentially attached to addresses via a standard version of serine and an artificially modified serine, respectively, and is also differentially modified with a post-translational moiety, thereby indicating the presence of the post-translational modification in the biological sample. The comparison also shows that the proteoform represented by the dotted bar attaches to an address in the first aliquot but not in the second aliquot, thereby indicating that the proteoform does not have any standard serines. The proteoform indicated by the hatched bar attaches to an address in the second aliquot but not in the first aliquot, thereby indicating that the proteoform does not have any post-translationally modified serines in the biological sample.

In further regard to the methods exemplified in the context of FIGS. 1A to 1E, and FIGS. 2A to 2D, proteoforms from separate aliquots can be attached to separate arrays or separate regions of an array. This can be useful for tracking the reactions to which each proteoform was subjected. In this configuration, individual proteoforms can each be uniquely identified by the site to which they are attached and the respective aliquot from which a given proteoform was derived can be distinguished by the array or array region to which the proteoform is attached. However, proteoforms from different aliquots can be distinguished using other configurations. For example, proteoforms from separate aliquots can be attached to separate sets of particles (e.g. different structured nucleic acid particles), respectively, and the different sets of particles can be distinguishable from each other. For example, a first set of particles can be distinguished from a second set of particles based on different physical attributes such as size, shape, charge or the like. The sets of particles can differ with respect to an attached label (e.g. luminophore or nucleic acid sequence). The physical attributes can be detectable in an array or can determine which sites in an array the particle will attach (e.g. a first set of sites in an array can be selective for one set of particles compared to a second set of sites that is selective for a second set of particles). Any of a variety of detectable attributes, such as those exemplified herein for unique identifiers and labels, can be used as a basis for distinguishing proteoforms of a first aliquot from proteoforms of a second aliquot.

As set forth above, a method of the present disclosure can be configured for differential treatment of a first aliquot of proteoforms compared to a second and differential modification of proteoforms between the two aliquots can be detected to facilitate identification of proteoforms or to characterize post-translational modifications in the proteoforms. In alternative configurations, differential modifications can occur within a sample and proteoforms that are differentially modified can be distinguished via differential tagging. For example, proteoforms that are modified in a first reaction can be attached to a first set of particles, whereas proteoforms that are not modified by the first reaction can be attached to a second set of particles, wherein the particles of the first set are detectably different, and thus distinguishable from, particles of the second set. Any of a variety of tagging schemes can be used including, for example, those exemplified herein for unique identifiers.

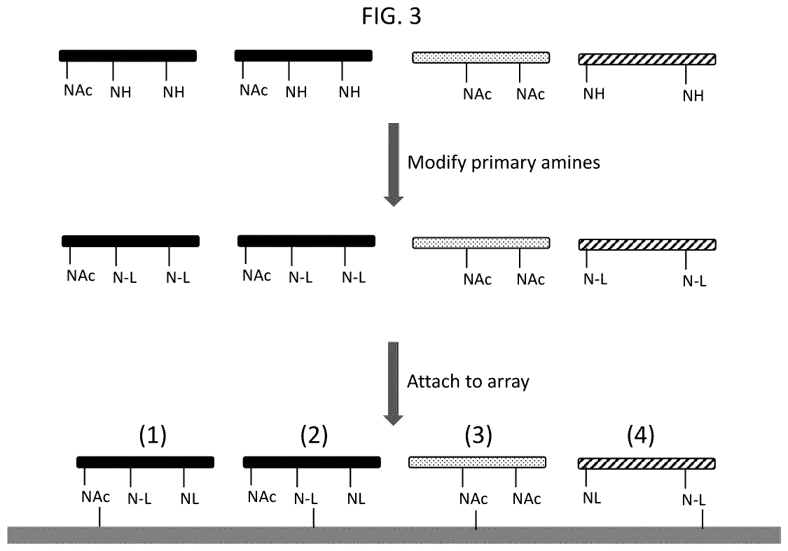

An exemplary configuration for differential modification of proteoforms within a sample is provided in FIG. 3. A sample having four proteoforms, two of which are identical, is subjected to a first reaction in which amines are modified. Free amines, such as those on lysine side chains, are modified by addition of an artificial moiety (L), but lysines having post-translationally added acetyl moieties (Ac) are not modified by addition of the artificial moiety. In the second reaction, the proteoforms are attached to an array having two different sets of sites. A first set of sites is reactive toward acetylated lysines and the sites react with proteoforms identified as (1) and (3). A second set of sites is reactive toward the artificial moiety and those sites react with proteoforms (2) and (4). The attachment reactions are orthogonal in this example and thus the two sets of sites provide tags for products of the respective reactions. The array can be subjected to a detection reaction that distinguishes artificially modified amino acids from post-translationally modified amino acids. For example, detection can use affinity reagents that selectively bind acetylated lysines compared to artificially modified lysines. Detection can also use affinity reagents that selectively bind artificially modified lysines compared to acetylated lysines. The results for proteoform (1) indicate that the original proteoform had an acetylated lysine (since the proteoform reacted with the array site), and standard lysines (since the proteoform binds to affinity reagents that recognize artificially modified lysines). The absence of binding by an affinity reagent for acetylated lysines indicates that proteoform (1) contained only a single acetylated lysine (i.e. the lysine that attaches proteoform (1) to the array). In this example, the affinity reagents do not recognize amino acids that have a bond to the array. The results for proteoform (2) indicate that the original proteoform had a standard lysine (since the proteoform reacted with the array site), acetylated lysines (since the proteoform binds to affinity reagents that recognize acetylated lysines), and more than one standard lysine (since the proteoform binds to affinity reagents that recognize artificially modified lysines). A protein identification assay can be performed to identify that the primary amino acid structures for proteoforms (1) and (2) are the same. Comparing the results of array attachment and probing with acetyl-specific and artificial moiety-specific affinity reagents indicates that the proteoforms have only one acetylated lysine and multiple artificially modified lysines. The results for proteoform (3) indicate that the original proteoform had multiple acetylated lysines (since the proteoform reacted with the array site and also bound to the affinity reagent that recognizes acetylated lysines.), and did not include standard lysines (since the proteoform did not bind to an affinity reagent that recognizes artificially modified lysines). The results for proteoform (4) indicate that the original proteoform had multiple standard lysines (since the proteoform reacted with the array site and also bound to the affinity reagent that recognizes artificially modified lysines), and did not include acetylated lysines (since the proteoform did not bind to an affinity reagent that recognizes acetylated lysines).

A method of the present disclosure can include a step of detecting a standard version of an amino acid by binding to an affinity reagent that selectively recognizes the standard version of the amino acid in comparison to an artificially modified version of the amino acid and to a post-translationally modified version of the amino acid. In some configurations of the methods, a post-translationally modified version of an amino acid can be detected by binding to an affinity reagent that selectively recognizes the post-translationally modified version of the amino acid in comparison to an artificially modified version of the amino acid and to a standard version of the amino acid. In yet other configurations, an artificially modified version of an amino acid can be detected by binding to an affinity reagent that selectively recognizes the artificially modified version of the amino acid in comparison to a post-translationally modified version of the amino acid and to a standard version of the amino acid.

Affinity reagents that distinguish post-translationally modified versions of amino acids from standard versions of the amino acids can be obtained, for example, from commercial sources. For example, antibodies having specificity for phosphotyrosine, phosphoserine, phosphothreonine, ubiquitin, SUMO, acylated lysine, and methyl lysine are commercially available from ThermoFisher (Waltham, MA). Antibodies having specificity for methylarginine, acetyllysine, carboxymethyllysine, carbamyllysine, phosphotyrosine, phosphoserine, phosphothreonine, ubiquitin, and SUMO are commercially available from Abcam (Cambridge, UK). Also useful are proteins and domains thereof that are referred to as “readers” for their ability to recognize specific post-translationally modified amino acids, for example, in specific amino acid sequence contexts. Such readers can be labeled and used as affinity reagents in a method set forth herein. Exemplary readers include histone readers such as those set forth in Musselman et al., Nat Struct Mol Biol. 19(12): 1218-1227 (2012), which is incorporated herein by reference. Another useful reader is the Src homology domain (SH2) which can bind phosphotyrosines. A modified version of the SH2 domain is capable of covalently attaching to phosphotyrosine proteins upon exposure to light as set forth in Ueza et al. Proc. Nat'l. Acad. Sci. USA E2929-E2938 (2012), which is incorporated herein by reference.

Chemoselective reagents can also be used to detect the presence or absence of a post-translational moiety on an amino acid. For example, phosphorimidazolide reagents can react selectively with phosphoryl groups. See Brown et al., ChemBioChem (2022) doi.org/10.1002/cbic.202200407, which is incorporated herein by reference.

### Proximity-Based Detection of Proteoforms

The present disclosure provides a method of detecting a proteoform based on proximity of two affinity reagents. For example, a first of two affinity reagents can bind to a post-translational moiety of a protein and a second of the two affinity reagents can bind to a sequence of standard amino acids (e.g. a dimer, trimer, tetramer, pentamer etc.) in the protein. In another example, a first of two affinity reagents can bind to a post-translational moiety of a protein and a second of the two affinity reagents can bind to an artificially modified amino acid in the protein. Interactions between the affinity reagents, when bound to the protein, can be detected whereby proximity is indicative of the presence of the post-translational moiety in the protein. The degree of proximity can also indicate structural characteristics of the protein such as conformational state.

Accordingly, a method of detecting a proteoform can include steps of (a) contacting an array of proteoforms with a first affinity reagent and a second affinity reagent, thereby forming a complex including the first affinity reagent bound to a post-translationally modified amino acid in a proteoform at an address of the array and the complex further including the second affinity reagent bound to an epitope in the proteoform, wherein the first affinity reagent is attached to a first nucleic acid and the second affinity reagent is attached to a second nucleic acid; (b) contacting the complex with a splint nucleic acid, wherein a first nucleotide sequence region of the splint nucleic acid hybridizes to the first nucleic acid and a second nucleotide sequence region of the splint nucleic acid hybridizes to the second nucleic acid; (c) detecting hybridization of the first and second nucleic acids to the splint nucleic acid, thereby detecting the proteoform at the address. Optionally, the epitope can be a sequence of standard amino acids or an artificially modified amino acid.

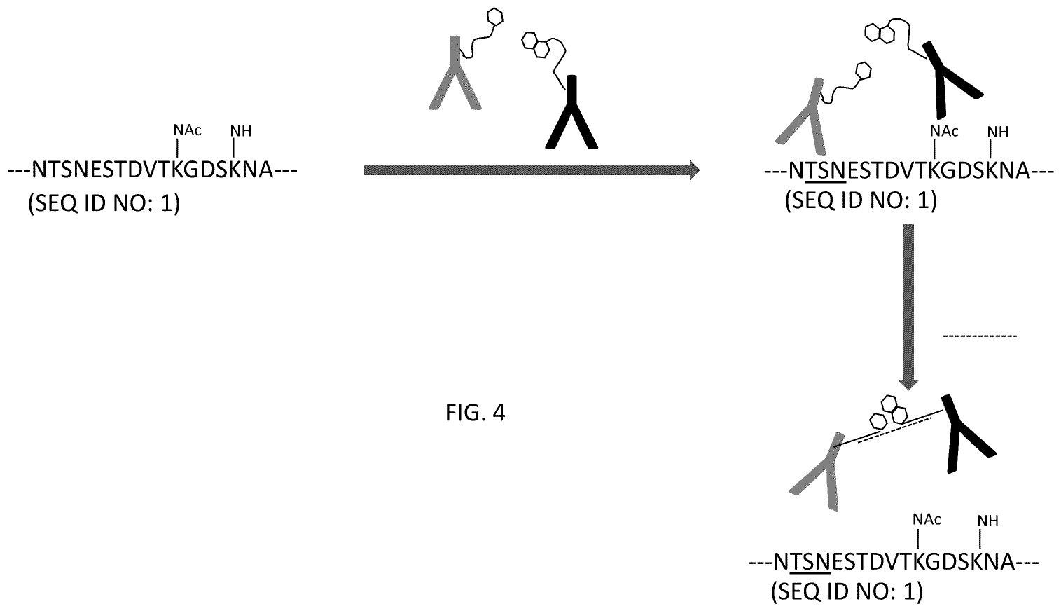

Exemplary assay steps are shown in FIG. 4. In the first step, a protein having an acetylated lysine in a region of its amino acid sequence (the region indicated using the IUPAC single amino acid code) and having a sequence NTSNESTDVTKGDSKNA (SEQ ID NO: 1) is bound to two affinity reagents to form a ternary complex. The ternary complex includes the protein, the first affinity reagent bound to the acetylated lysine and the second affinity reagent bound to the TSN trimer epitope. To each of the affinity reagents is attached a nucleic acid tag having a luminophore. The luminophores on the respective nucleic acid tags are capable of Förster resonance energy transfer (FRET). However, efficiency of FRET is low if the luminophores are not oriented in proximity to each other. FRET can be facilitated via the second step shown in the figure, whereby a splint oligonucleotide is added to the complex. The splint oligonucleotide includes a first nucleotide sequence region that is complementary to the nucleic acid tag of the first affinity reagent and also include a second nucleotide sequence region that complements the nucleic acid tag of the second affinity reagent. If the two affinity reagents are in sufficient proximity to each other when in the ternary complex, then the splint oligonucleotide can hybridize to their respective nucleic acid tags to orient the luminophores for FRET. As such, detection of FRET indicates presence of the two binding sites (acetylated lysine and TSN, respectively) in the protein and in proximity to each other. Absence of FRET indicates the absence of proximal sites. In the latter case, the complex can be measured to determine if one or both of the luminophores is present. Presence of both luminophores, absent FRET, indicates that an acetylated lysine and TSN epitope are both present in the protein despite not being sufficiently proximal for the affinity reagents to interact. Absence of one or both of the luminophores indicates absence of one or both of the acetylated lysine or TSN in the protein.

Any of a variety of tags can be used to detect proximity of two affinity reagents. Nucleic acids are particularly useful tags for the affinity reagents. The well understood correlation between nucleotide sequence and hybridization strength can be exploited to tailor nucleic acid tags for interactions that are strong enough to facilitate detection, but not so strong as to substantially alter binding of affinity reagents to their respective epitopes in a protein. Nucleic acid synthesis techniques are well established for synthesizing nucleic acids in a wide range of lengths, for example, at least about 5, 10, 15, 20, 25, 50, 100 or more nucleotides in length. Alternatively or additionally, nucleic acid tags can be no more than 100, 50, 25, 20, 15, 10, 5 or fewer nucleotides in length. A wide variety of artificial moieties, such as label moieties, binding moieties, and reactive moieties, can be incorporated into nucleic acids at specified sequence positions using well established synthesis techniques. Moreover, the relative flexibility of nucleic acids can accommodate interactions between affinity reagents that are in a wide variety of relative spatial orientations.

Interactions between nucleic acid tags can be detected in any of a variety of ways. As exemplified in FIG. 4, nucleic acid tags from respective affinity reagents can be oriented by a splint oligonucleotide such that luminophores on the tags engage in FRET. However, FRET need not be used. Instead, the oligonucleotide tags can bind to a labeled splint oligonucleotide such that detection of the label indicates proximity of the two nucleic acid tags. In this case, the conditions for splint oligonucleotide hybridization can be sufficiently stringent that both nucleic acid tags must be present for the splint to be detectably retained in the ternary complex. In other configurations, hybridization of a splint oligonucleotide can position the nucleic acid tags for enzymatic modification. For example, a ligase can be contacted with the hybrid such that the nucleic acid tags ligate to each other, thereby attaching the probes to each other. The attached probes can be detected, for example, after removal from the complex. The nucleotide sequence formed by the ligated nucleic acids can be detected, for example, using real time polymerase chain reaction, nucleotide sequencing or other nucleic acid detection techniques.

Splint oligonucleotides need not be used to detect proximity of nucleic acid tagged affinity reagents. For example, the nucleic acids can include sequence regions that are complementary to each other. Again, the conditions for hybridization can be sufficiently stringent that the nucleic acid tags hybridize after the respective affinity reagents have formed a ternary complex with the protein but interaction between the tags is too weak to occur stably before the ternary complex forms. Hybridization between the tags can be detected by FRET between luminophores on the respective tags. Also useful is bioluminescence resonance energy transfer (BRET). BRET can be configured to exploit the naturally occurring phenomenon of dipole-dipole energy transfer from a donor enzyme (e.g. luciferase) to an acceptor fluorophore following enzyme-mediated oxidation of a substrate. See Dale et al., Front. Bioeng. and Biotech. Vol. 7-2019 doi.org/10.3389/fbioc.2019.00056, which is incorporated herein by reference. An enzymatic assay can also be used to detect the hybridized tags, including for example, addition of a labeled nucleotide to the 3′ end of one tag using a polymerase that reads a given nucleotide in the template provided by the other tag.

### Multivalent Affinity Reagents

The present disclosure provides a method of detecting a proteoform using a multivalent affinity reagent. A multivalent affinity reagent can include two or more different affinity moieties. The affinity moieties of a multivalent affinity reagent can differ in both structure and function such that one affinity moiety of the reagent recognizes a first epitope but not a second epitope, and another affinity moiety of the reagent recognizes the second epitope but not the first epitope. Particularly useful multivalent affinity reagents have a first affinity moiety that recognizes a post-translational moiety of a protein and a second affinity moiety that recognizes a second epitope in the protein. The second epitope is different from the post-translational moiety and can be, for example, a different post-translational moiety, an artificial moiety or a sequence of standard amino acids (e.g. a dimer, trimer, tetramer, pentamer etc.). In some cases, the first and second affinity moieties can recognize the same post-translational moiety albeit on different amino acids or in different amino acid sequence contexts. In other cases, the first and second affinity moieties can recognize the same artificial moiety albeit on different amino acids or in different amino acid sequence contexts.

Accordingly, a method of detecting a proteoform, can include steps of (a) contacting an array of proteoforms with a multivalent affinity reagent, thereby forming a complex including a first affinity moiety of the multivalent affinity reagent bound to a post-translationally modified amino acid of a proteoform at an address of the array, the complex further including a second affinity moiety of the multivalent affinity reagent bound to an epitope (e.g. a sequence of standard amino acids, an artificial moiety or another post-translationally modified amino acid) in the proteoform; and (b) detecting the complex at the address.

Two or more affinity moieties of a multivalent affinity reagent can be attached to each other via a retaining component. The retaining component can be polymeric in nature, for example, being composed of protein, nucleic acid or artificial polymer. Particularly useful retaining components are structured nucleic acid particles such as nucleic acid origami. Antibody structures also provide useful retaining components for recombinant affinity reagents having different binding sites (i.e. different affinity moieties). Exemplary antibody structures include nanobodies. See, for example, Wang et al., Front. Immunol., Sec. Vaccines and Molecular Therapeutics “Research Progress and Applications of Multivalent, Multispecific and Modified Nanobodies for Disease Treatment” 12-2021 (2022), doi.org/10.3389/fimmu.2021.838082, which is incorporated herein by reference. Particles, such as beads or structured nucleic acid particles (e.g. nucleic acid origami) provide particularly useful retaining components. These and other useful retaining components are set forth in US Pat. App. Pub. No. 2022/0162684 A1, which is incorporated herein by reference.

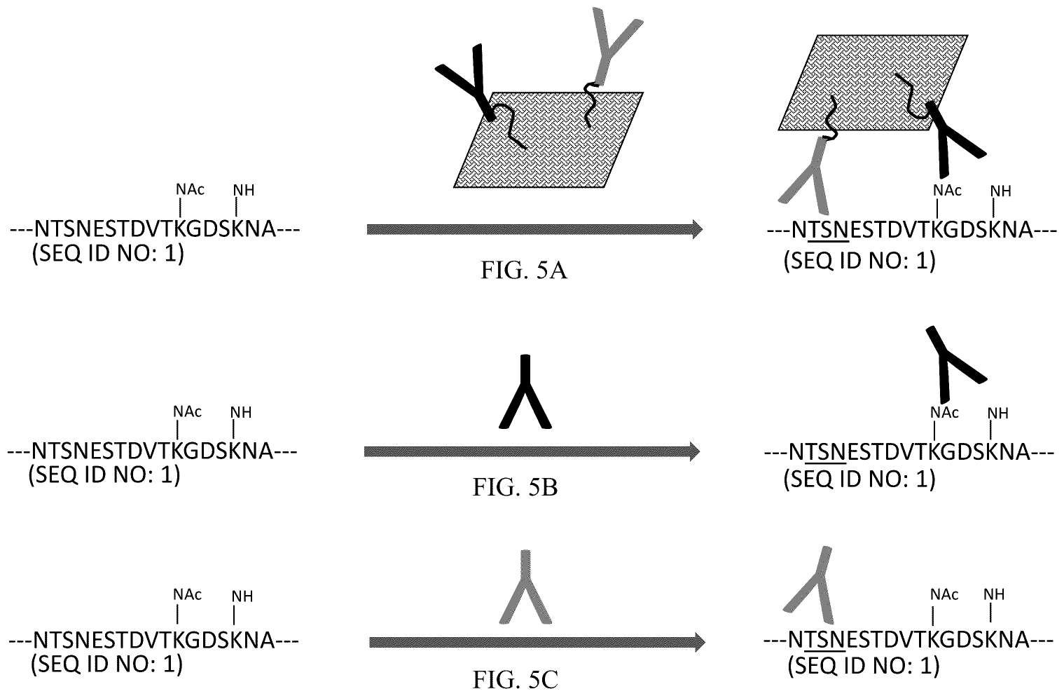

A method of the present disclosure can be configured to detect binding of a multivalent affinity reagent, having two or more different affinity moieties, compared to binding of a monovalent affinity reagent having one of the affinity moieties. FIG. 5A shows a multivalent probe having a first affinity moiety that binds to an acetylated lysine epitope on an amino acid sequence NTSNESTDVTKGDSKNA (SEQ ID NO: 1) and a second affinity moiety that binds to an epitope consisting of a trimer of standard amino acids (i.e. TSN). The two epitopes are sufficiently proximal in the protein that both affinity moieties can bind simultaneously to the protein. In this example, the affinity moieties are antibodies (or fragments thereof) attached to a nucleic acid origami tile (indicated by a parallelogram). As shown in FIG. 5B and FIG. 5C, the two antibodies can also be separately contacted with the protein (or a copy of the protein). Differences in the extent of binding for the multivalent affinity reagent compared to one or both of the monovalent affinity reagents can be measured and analyzed to determine the presence or absence of an epitope that is recognized by one of the affinity moieties.

In some configurations, a method of detecting a proteoform, can include steps of (a) contacting an array of proteoforms with a multivalent affinity reagent, thereby forming a complex including a first affinity moiety of the multivalent affinity reagent bound to a post-translationally modified amino acid of a proteoform at an address of the array, the complex further including a second affinity moiety of the multivalent affinity reagent bound to an epitope (e.g. a sequence of standard amino acids, an artificial moiety or another post-translationally modified amino acid) in the proteoform; (b) detecting the complex at the address; (c) contacting the array of proteoforms with a monovalent affinity reagent, wherein (i) the monovalent affinity reagent comprises the first affinity moiety and lacks the second affinity moiety, or (ii) the monovalent affinity reagent comprises the second affinity moiety and lacks the first affinity moiety, thereby forming a second complex at the address, and (d) detecting the second complex at the address. Optionally, the multivalent affinity reagent and the monovalent affinity reagent are not simultaneously present at the address. However, the multivalent affinity reagent and the monovalent affinity reagent can be simultaneously present at the address, if desired. Moreover, the multivalent affinity reagent can be contacted with the array before or after contacting the array with the monovalent affinity reagent. Optionally, the array can be washed between the steps of contacting the affinity reagents with the array, thereby removing residual reagents.

The above method can further include a step of identifying the post-translationally modified amino acid of the proteoform based on higher affinity of the multivalent affinity reagent to the proteoform compared to affinity of the monovalent affinity reagent to the proteoform. Alternatively or additionally, the above method can include a step of identifying absence of the post-translationally modified amino acid of the proteoform based on similar affinity of the multivalent affinity reagent to the proteoform compared to affinity of the monovalent affinity reagent to the proteoform.

In some configurations of the above method, the array can include a plurality of addresses having proteoforms with the same amino acid sequence as the proteoform at the address, and the level of binding can be measured as (i) the number of addresses in the plurality of addresses that bind to the multivalent affinity reagent and to the monovalent affinity reagent, (ii) the rate of binding for the multivalent affinity reagent and the monovalent affinity reagent at one or more addresses in the plurality of addresses, or (iii) the rate of dissociation for the multivalent affinity reagent and the monovalent affinity reagent from one or more addresses in the plurality of addresses.

### Biological Samples

One or more proteins that are used in a method, composition or apparatus herein, can be derived from a natural or synthetic source. Exemplary sources include, but are not limited to biological tissue, fluid, cells or subcellular compartments (e.g. organelles). For example, a sample can be derived from a tissue biopsy, biological fluid (e.g. blood, plasma, extracellular fluid, urine, mucus, saliva, semen, vaginal fluid, sweat, synovial fluid, lymph, cerebrospinal fluid, peritoneal fluid, pleural fluid, amniotic fluid, intracellular fluid, extracellular fluid, etc.), fecal sample, hair sample, cultured cell, culture media, fixed tissue sample (e.g. fresh frozen or formalin-fixed paraffin-embedded) or protein synthesis reaction. A protein source may comprise any sample where a protein is a native or expected constituent. For example, sources for gastric enzymes may include cells from digestive organs, sample from gastric ducts, or fluid samples from digestive organs (e.g., bile). In a second example, a primary source for a cancer biomarker protein may be a tumor biopsy sample. Other sources include environmental samples or forensic samples.

Exemplary organisms from which one or more proteins can be derived include, for example, a mammal such as a rodent, mouse, rat, rabbit, guinea pig, ungulate, horse, sheep, pig, goat, cow, cat, dog, primate, non-human primate or human; a plant such as Arabidopsis thaliana, tobacco, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish; a reptile; an amphibian such as a frog or Xenopus laevis; a dictyostelium discoideum; a fungi such as Pneumocystis carinii, Takifugu rubripes, yeast, Saccharamoyces cerevisiae or Schizosaccharomyces pombe; or a Plasmodium falciparum. Proteins can also be derived from a prokaryote such as a bacterium, Escherichia coli, staphylococci or Mycoplasma pneumoniae; an archae; a virus such as Hepatitis C virus, influenza virus, coronavirus, or human immunodeficiency virus; or a viroid. Proteins can be derived from a homogeneous culture or population of the above organisms or alternatively from a collection of several different organisms, for example, in a community or ecosystem.

In some cases, one or more proteins can be derived from an organism that is collected from a host organism. One or more proteins may be derived from a parasitic, pathogenic, symbiotic, or latent organism collected from a host organism. Protein(s) can be derived from an organism, tissue, cell or biological fluid that is known or suspected of being linked with a disease state or disorder (e.g., an oncogenic virus). Alternatively, protein(s) can be derived from an organism, tissue, cell or biological fluid that is known or suspected of not being linked to a particular disease state or disorder. For example, protein(s) isolated from such a source can be used as a control for comparison to results acquired from a source that is known or suspected of being linked to the particular disease state or disorder. A sample may comprise a microbiome. A sample may comprise a plurality of proteins contributed by microbiome constituents. In some cases, one or more proteins used in a method, composition or apparatus set forth herein may be obtained from a single organism (e.g. an individual human), single cell, single organelle, or single protein-containing particle (e.g., a viral particle).

A plurality of proteins can be characterized in terms of total protein mass. For example, plurality of proteins used or included in a method, composition or apparatus set forth herein can include at least 1 pg, 10 pg, 100 pg, 1 ng, 10 ng, 100 ng, 1 μg, 10 μg, 100 μg, 1 mg, 10 mg, 100 mg or more protein by mass. Alternatively or additionally, a plurality of proteins may contain at most 100 mg, 10 mg, 1 mg, 100 μg, 10 μg, 1 μg, 100 ng, 10 ng, 1 ng, 100 pg, 10 pg, 1 pg or less protein by mass.

A method, composition or apparatus of the present disclosure can use or include a proteomic sample. A proteomic sample can include substantially all proteins from a given source or a substantial fraction thereof. For example, a proteomic sample may contain at least 60%, 75%, 90%, 95%, 99%, 99.9% or more of the total protein mass present in the source from which the sample was derived. Alternatively or additionally, a proteomic sample may contain at most 99.9%, 99%, 95%, 90%, 75%, 60% or less of the total protein mass present in the source from which the sample was derived.

A plurality of proteins can be characterized in terms of total number of protein molecules. A plurality of proteins used or included in a method, composition or apparatus set forth herein can include at least 1 protein molecule, 10 protein molecules, 100 protein molecules, 1×10⁴ protein molecules, 1×10⁶ protein molecules, 1×10⁸ protein molecules, 1×10¹⁰ protein molecules, 1 mole (6.02214076×10²³ molecules) of protein, 10 moles of protein molecules, 100 moles of protein molecules or more. Alternatively or additionally, a plurality of proteins may contain at most 100 moles of protein molecules, 10 moles of protein molecules, 1 mole of protein molecules, 1×10¹⁰ protein molecules, 1×10⁸ protein molecules, 1×10⁶ protein molecules, 1×10⁴ protein molecules, 100 protein molecules, 10 protein molecules, 1 protein molecule or less.

A plurality of proteins can be characterized in terms of the variety of full-length primary protein structures in the plurality. For example, the variety of full-length primary protein structures in a plurality of proteins can be equated with the number of different protein-encoding genes in the source for the plurality of proteins. Whether or not the proteins are derived from a known genome or from any genome at all, the variety of full-length primary protein structures can be counted independent of presence or absence of post-translational modifications in the proteins. A plurality of proteins can have a complexity that includes substantially all different native-length protein primary sequences from a given source. A proteome or subfraction can have a complexity of at least 2, 5, 10, 100, 1×10³, 1×10⁴, 2×10⁴, 3×10⁴ or more different native-length protein primary sequences. Alternatively or additionally, a proteome or subfraction can have a complexity that is at most 3×10⁴, 2×10⁴, 1×10⁴, 1×10³, 100, 10, 5, 2 or fewer different native-length protein primary sequences.

The diversity of a proteomic sample can include at least one representative for substantially all proteins encoded by a source from which the sample was derived or a substantial fraction thereof. For example, a proteomic sample may contain at least one representative for at least 60%, 75%, 90%, 95%, 99%, 99.9% or more of the proteins encoded by a source from which the sample was derived. Alternatively or additionally, a proteomic sample may contain a representative for at most 99.9%, 99%, 95%, 90%, 75%, 60% or less of the proteins encoded by a source from which the sample was derived.

A plurality of proteins can be characterized in terms of the variety of protein structures in the plurality including different primary structures and different proteoforms among the primary structures. Different molecular forms of proteins expressed from a given gene are considered to be different proteoforms. Proteoforms can differ, for example, due to differences in primary structure (e.g. shorter or longer amino acid sequences), different arrangement of domains (e.g. transcriptional splice variants), or different post-translational modifications (e.g. presence or absence of phosphoryl, glycosyl, acetyl, or ubiquitin moieties). A plurality of proteins used or included in a method, composition or apparatus set forth herein can have a complexity of at least 2, 5, 10, 100, 1×10³, 1×10⁴, 1×10⁵, 1×10⁶, 5×10⁶, 1×10⁷ or more different protein structures. Alternatively or additionally, a plurality of proteins can have a complexity that is at most 1×10⁷, 5×10⁶, 1×10⁶, 1×10⁵, 1×10⁴, 1×10³, 100, 10, 5, 2 or fewer different protein structures.

A plurality of proteins can be characterized in terms of the dynamic range for the different protein structures in the plurality. The dynamic range can be a measure of the range of abundance for all different protein structures in a plurality of proteins, the range of abundance for all different primary protein structures in a plurality of proteins, the range of abundance for all different full-length primary protein structures in a plurality of proteins, the range of abundance for all different full-length gene products in a plurality of proteins, the range of abundance for all different proteoforms expressed from a given gene, or the range of abundance for any other set of different proteins set forth herein. The dynamic range for plurality of proteins set forth herein can be a factor of at least 10, 100, 1×10³, 1×10⁴, 1×10⁶, 1×10⁸, 1×10¹⁰, or more. Alternatively or additionally, the dynamic range for plurality of proteins set forth herein can be a factor of at most 1×10¹⁰, 1×10⁸, 1×10⁶, 1×10⁴, 1×10³, 100, 10 or less.

A sample can include different proteoforms of a particular protein. For example, at least 1, 2, 3, 4, 5, 10, 15, 20, 25 or more proteoforms from a particular gene can be present in a method, composition or apparatus set forth herein. Alternatively or additionally, at most 25, 20, 15, 10, 5, 4, 3, 2 or 1 proteoforms from a particular gene can be present in a method, composition or apparatus set forth herein. A method set forth herein can be configured to distinguish the proteoforms. For example, proteoforms can be distinguished with regard to differences in the presence, location or type of post-translational modifications occurring at least at 2, 3, 4, 5, 10, 15, 20, 25 or more residues of a particular amino acid sequence that is shared by the proteoforms. Alternatively or additionally, proteoforms can be distinguished with regard to the presence, location or type of post-translational modifications occurring at most at 25, 20, 15, 10, 5, 4, 3, 2 or 1 residues of a particular amino acid sequence that is shared by the proteoforms.

One or more proteins can optionally be separated or isolated from other components of the source for the protein(s). For example, one or more proteins can be separated or isolated from lipids, nucleic acids, hormones, enzyme cofactors, vitamins, metabolites, microtubules, organelles (e.g. nucleus, mitochondria, chloroplast, endoplasmic reticulum, vesicle, cytoskeleton, vacuole, lysosome, cell membrane, cytosol or Golgi apparatus) or the like. Protein separation can be carried out using methods known in the art such as centrifugation (e.g. to separate membrane fractions from soluble fractions), density gradient centrifugation (e.g. to separate different types of organelles), precipitation, affinity capture (e.g. to capture post-translationally modified proteins using immobilized affinity agents having specificity for post-translational modifications), adsorption, liquid-liquid extraction, solid-phase extraction, chromatography (e.g. affinity chromatography, ion exchange chromatography, reverse phase chromatography, size exclusion chromatography, electrophoresis (e.g. polyacrylamide gel electrophoresis) or the like. Particularly useful protein separation methods are set forth in Scopes, Protein Purification Principles and Practice, Springer; 3rd edition (1993). In particular configurations of the methods set forth herein, a protein sample can be enriched for proteoforms of a particular type. For example, proteoforms having a particular post-translational modification can be enriched by affinity capture and removal of proteoforms lacking the post-translational modification. Such enrichment can occur for proteins prior to being subjected to capture, labelling or detection methods set forth herein. Any of a variety of affinity reagents set forth herein can be immobilized on a solid support and used to capture proteoforms of interest.

In some configurations of the methods, compositions or apparatus set forth herein, proteins can be in a native state, for example, being capable of performing native function(s) such as catalysis of reactions. Alternatively, proteins can be in a denatured state, for example, being incapable of performing native function(s) such as catalysis of reactions. One or more proteins can be in a native state for some manipulations set forth herein and in a non-native state for other manipulations set forth herein. Protein(s) may be denatured at any stage during manipulation, including for example, upon removal from a native milieu or at a later stage of processing such as a stage where protein(s) are separated from other cellular components, fractionated from other proteins, labelled, attached to a solid support, attached to a unique identifier, contacted with a binding reagent, detected or other step set forth herein. Denatured proteins may be refolded, for example, reverting to a native state for one or more step of a process set forth herein.

### Protein Modifications

Methods of the present disclosure are particularly well suited for manipulating and detecting proteoforms. The methods can be used to differentially manipulate proteoforms based on unique molecular properties or to distinguish one proteoform from another.

Proteoforms can differ with regard to presence or absence of a post-translational modification, type of post-translational modification present, location of a post-translational modification, number of post-translational modifications present or combination thereof. A post-translational modification may be one or more of myristoylation, palmitoylation, isoprenylation, prenylation, farnesylation, geranylgeranylation, lipoylation, flavin moiety attachment, Heme C attachment, phosphopantetheinylation, retinylidene Schiff base formation, dipthamide formation, ethanolamine phosphoglycerol attachment, hypusine, beta-Lysine addition, acylation, acetylation, deacetylation, formylation, alkylation, methylation, C-terminal amidation, arginylation, polyglutamylation, polyglyclyation, butyrylation, gamma-carboxylation, glycosylation, glycation, polysialylation, malonylation, hydroxylation, iodination, nucleotide addition, phosphoate ester formation, phosphoramidate formation, phosphorylation, adenylylation, uridylylation, propionylation, pyrolglutamate formation, S-glutathionylation, S-nitrosylation, S-sulfenylation, S-sulfinylation, S-sulfonylation, succinylation, sulfation, glycation, carbamylation, carbonylation, isopeptide bond formation, biotinylation, carbamylation, oxidation, reduction, pegylation, ISGylation, SUMOylation, ubiquitination, neddylation, pupylation, citrullination, deamidation, elminylation, disulfide bridge formation, isoaspartate formation, and racemization.

A post-translational modification may occur at a particular type of amino acid residue in a protein. For example, the phosphate moiety of a particular proteoform can be present on a serine, threonine, tyrosine, histidine, cysteine, lysine, aspartate or glutamate residue. In another example, an acetyl moiety of a particular proteoform can be present on the N-terminus or on a lysine of a protein. In another example, a serine or threonine residue of a proteoform can have an O-linked glycosyl moiety, or an asparagine residue of a proteoform can have an N-linked glycosyl moiety. In another example, a proline, lysine, asparagine, aspartate or histidine amino acid of a proteoform can be hydroxylated. In another example, a proteoform can be methylated at an arginine or lysine amino acid. In another example, a proteoform can be ubiquitinated at the N-terminal methionine or at a lysine amino acid.

A post-translationally modified versions of a given amino acid can include a post-translational moiety at a side chain position that is unmodified in a standard version of the amino acid. Post-translationally modified lysines can include epsilon amines attached to post-translational moieties, whereas standard lysines have epsilon amines lacking the post-translational moieties. Post-translationally modified histidines can include side-chain tertiary amines attached to post-translational moieties, whereas in standard histidines the side-chain amines are secondary amines lacking the post-translational moieties. Post-translationally modified versions of aspartates or glutamates can include side-chain carbonyls, esters or amides attached to post-translational moieties, whereas in standard versions of aspartates or glutamates the side-chains have carboxyls lacking the post-translational moieties. Post-translationally modified versions of arginines can include side-chain amines attached to post-translational moieties, whereas in standard versions of arginines the side-chain amines lack the post-translational moieties. Post-translationally modified versions of cysteines can include thioethers attached to post-translational moieties, whereas standard versions of cysteines have sulfurs lacking the post-translational moieties. Post-translationally modified versions of serines, threonines or tyrosines can include ethers or esters attached to post-translational moieties, whereas standard versions of serines, threonines or tyrosines have hydroxyls lacking the post-translational moieties.

### Protein Assays

A protein can be detected using one or more affinity reagents having binding affinity for the protein. An affinity reagent can bind to a protein to form a complex and the complex can be detected. The complex can be detected directly, for example, due to a label that is present on the affinity reagent or protein. In some configurations, the complex need not be directly detected, for example, in formats where the complex is formed and then the affinity reagent, protein, or a label component that was present in the complex is subsequently detected.

Any of a variety of affinity reagents can be used to detect proteins based on their amino acid sequences. Any molecule or other substance that is capable of specifically or reproducibly binding to a protein can be used as an affinity reagent. An affinity reagent can be larger than, smaller than or the same size as the protein to which it binds. An affinity reagent may form a reversible or irreversible bond with a protein. An affinity reagent may bind with a protein in a covalent or non-covalent manner. Affinity reagents may include reactive affinity reagents, catalytic affinity reagents (e.g., kinases, proteases, etc.) or non-reactive affinity reagents (e.g., antibodies or fragments thereof). An affinity reagent can be non-reactive and non-catalytic, thereby not permanently altering the chemical structure of a protein to which it binds. Affinity reagents that can be particularly useful for binding to proteins include, but are not limited to, antibodies or functional fragments thereof (e.g., Fab′ fragments, F(ab′)₂ fragments, single-chain variable fragments (scFv), di-scFv, tri-scFv, or microantibodies), affibodies, affilins, affimers, affitins, alphabodies, anticalins, avimers, DARPins, monobodies, nanoCLAMPs, lectins or functional fragments thereof.

An affinity reagent can include a label. Exemplary labels include, without limitation, a luminophore (e.g. fluorophore), chromophore, nanoparticle (e.g., gold, silver, carbon nanotubes), heavy atom, radioactive isotope, mass label, charge label, spin label, receptor, ligand, nucleic acid barcode, polypeptide barcode, polysaccharide barcode, or the like. A label can produce any of a variety of detectable signals including, for example, an optical signal such as absorbance of radiation, luminescence (e.g. fluorescence or phosphorescence) emission, luminescence lifetime, luminescence polarization, or the like; Rayleigh and/or Mie scattering; magnetic properties; electrical properties; charge; mass; radioactivity or the like. A label may produce a signal with a characteristic frequency, intensity, polarity, duration, wavelength, sequence, or fingerprint. A label need not directly produce a signal. For example, a label can bind to a receptor or ligand having a moiety that produces a characteristic signal. Such labels can include, for example, nucleic acids that are encoded with a particular nucleotide sequence, avidin, biotin, non-peptide ligands of known receptors, or the like.

Many protein assays, such as enzyme linked immunosorbent assay (ELISA), achieve high-confidence characterization of one or more proteins in a sample by exploiting high specificity binding of affinity reagents to the polypeptide(s) and detecting the binding event while ignoring all other proteins in the sample. Binding assays can be carried out by detecting affinity reagents and/or proteins that are immobilized in multiwell plates, on arrays, or on particles in microfluidic devices. Exemplary plate-based methods include, for example, the MULTI-ARRAY technology commercialized by MesoScale Diagnostics (Rockville, Maryland) or Simple Plex technology commercialized by Protein Simple (San Jose, CA). Exemplary, array-based methods include, but are not limited to those utilizing Simoa® Planar Array Technology or Simoa® Bead Technology, commercialized by Quanterix (Billerica, MA). Further exemplary array-based methods are set forth in U.S. Pat. Nos. 9,678,068; 9,395,359; 8,415,171; 8,236,574; or 8,222,047, each of which is incorporated herein by reference. Exemplary microfluidic detection methods include those commercialized by Luminex (Austin, Texas) under the trade name xMAP® technology or used on platforms identified as MAGPIX®, LUMINEX® 100/200 or FEXMAP 3D®).

Exemplary assay formats that can be performed at a variety of plexity scales, up to and including proteome scale, are set forth in U.S. Pat. No. 10,473,654 or US Pat. App. Pub. Nos. 2020/0318101 A1 or 2020/0286584 A1; U.S. patent application Ser. No. 18/045,036, or Egertson et al., BioRxiv (2021), DOI: 10.1101/2021.10.11.463967, each of which is incorporated herein by reference. A plurality of proteins can be assayed for binding to affinity reagents, for example, on single-molecule resolved protein arrays. Proteins can be in a denatured state or native state when manipulated or detected in a method set forth herein.

Turning to the example of an array-based assay configuration, the identity of an extant protein at any given address is typically not known prior to performing the assay. The assay can be used to identify extant proteins at one or more addresses in the array. A plurality of affinity reagents, optionally labeled (e.g. with fluorophores), can be contacted with the array, and the presence of affinity reagents can be detected at individual addresses to determine binding outcomes. A plurality of different affinity reagents can be delivered to the array and detected serially, such that each cycle detects binding outcomes for a given type of affinity reagent (e.g. a type of affinity reagent having affinity for a particular epitope) at each address. The assay can include a step of dissociating affinity reagents from the array after detecting the binding outcomes, such that the next affinity reagent can be delivered and detected. In some configurations, a plurality of different affinity reagents can be detected in parallel, for example, when different affinity reagents are distinguishably labeled.

A protein, for example at an address of an array, can be contacted with a plurality of different affinity reagents. For example, a plurality of affinity reagents (whether configured separately or as a pool) may include at least 2, 5, 10, 25, 50, 100, 250, 500 or more types of affinity reagents, each type of affinity reagent differing from the other types with respect to the epitope(s) recognized. Alternatively or additionally, a plurality of affinity reagents may include at most 500, 250, 100, 50, 25, 10, 5, or 2 types of affinity reagents. Different types of affinity reagents in a pool can be uniquely labeled such that the different types can be distinguished from each other. In some configurations, at least two, and up to all, of the different types of affinity reagents in a pool may be indistinguishably labeled. Alternatively or additionally to the use of unique labels, different types of affinity reagents can be delivered and detected serially when evaluating a protein.

In particular configurations, a method set forth herein can be used to identify a number of different extant proteins that exceeds the number of affinity reagents used. For example, the number of different protein species identified can be at least 5×, 10×, 25×, 50×, 100× or more than the number of affinity reagents used. This can be achieved, for example, by (1) using promiscuous affinity reagents that bind to multiple different candidate proteins suspected of being present in a given sample, and (2) subjecting the extant proteins to a set of promiscuous affinity reagents that, taken as a whole, are expected to bind each candidate protein in a different combination, such that each candidate protein is expected to generate a unique profile of binding and non-binding events. Promiscuity of an affinity reagent can arise due to the affinity reagent recognizing an epitope that is known to be present in a plurality of different candidate proteins. For example, epitopes having relatively short amino acid lengths such as dimers, trimers, tetramers or pentamers can be expected to occur in a substantial number of different proteins in a typical proteome. Alternatively or additionally, a given promiscuous affinity reagent may recognize multiple different epitopes (e.g. epitopes differing from each other with regard to amino acid composition or sequence). For example, a promiscuous affinity reagent that is designed or selected for its affinity toward a first trimer epitope may also have affinity for a second epitope that has a different sequence of amino acids compared to the first epitope.

Although performing a single binding reaction between a promiscuous affinity reagent and a complex protein sample may yield ambiguous results regarding the identity of the different extant proteins to which it binds, the ambiguity can be resolved by decoding the binding profiles for each extant protein using machine learning or artificial intelligence algorithms that are based on probabilities for the affinity reagents binding to candidate proteins. For example, a plurality of different promiscuous affinity reagents can be contacted with a complex population of extant proteins, wherein the plurality is configured to produce a different binding profile for each candidate protein suspected of being present in the population. The plurality of promiscuous affinity reagents can produce a binding profile for each extant protein that can be decoded to identify a unique combination of positive outcomes (i.e. observed binding events) and/or negative binding outcomes (i.e. observed non-binding events), and this can in turn be used to identify the extant protein as a particular candidate protein having a high likelihood of exhibiting a similar binding profile.

Binding profiles can be obtained for extant proteins and decoded. In many cases one or more binding events produces inconclusive or even aberrant results and this, in turn, can yield ambiguous binding profiles. For example, observation of binding outcome at single-molecule resolution can be particularly prone to ambiguities due to stochasticity in the behavior of single molecules when observed using certain detection hardware. As set forth above, ambiguity can also arise from affinity reagent promiscuity. Decoding can utilize a binding model that evaluates the likelihood or probability that one or more candidate proteins that are suspected of being present in an assay will have produced an empirically observed binding profile. The binding model can include information regarding expected binding outcomes (e.g. positive binding outcomes and/or negative binding outcomes) for one or more affinity reagents with respect to one or more candidate proteins. A binding model can include a measure of the probability or likelihood of a given candidate protein generating a false positive or false negative binding result in the presence of a particular affinity reagent, and such information can optionally be included for a plurality of affinity reagents.

Decoding can be configured to evaluate the degree of compatibility of one or more empirical binding profiles with results computed for various candidate proteins using a binding model. For example, to identify an extant protein in a sample, an empirical binding profile for the extant protein can be compared to results computed by the binding model for many or all candidate proteins suspected to be in the sample. A machine learning or artificial intelligence algorithm can be used. An algorithm used for decoding can utilize Bayesian inference. In some configurations, identity for an extant protein is determined based on a likelihood of the extant protein being a particular candidate protein given the empirical binding pattern or based on the probability of a particular candidate protein generating the empirical binding pattern. Particularly useful decoding methods are set forth, for example, in U.S. Pat. No. 10,473,654; US Pat. App. Pub. No. 2020/0318101 A1; U.S. patent application Ser. No. 18/045,036, or Egertson et al., BioRxiv (2021), DOI: 10.1101/2021.10.11.463967, each of which is incorporated herein by reference.

A protein assay can employ cyclical modification of proteins and the modified products from individual cycles can be detected. The proteins can be identified based on the results. In some configurations, a protein can be sequenced by a sequential process in which each cycle includes steps of detecting the protein and removing one or more terminal amino acids from the protein. Optionally, one or more of the steps can include adding a label to the protein, for example, at the amino terminal amino acid or at the carboxy terminal amino acid. In particular configurations, an assay for detecting a protein can include steps of (i) exposing a terminal amino acid on the protein; (ii) detecting a change in signal from the protein; and (iii) identifying the type of amino acid that was removed based on the change detected in step (ii). The terminal amino acid can be exposed, for example, by removal of one or more amino acids from the amino terminus or carboxyl terminus of the protein. Steps (i) through (iii) can be repeated to produce a series of signal changes that is indicative of the sequence for the protein.

In a first configuration of a cyclical protein detection assay, one or more types of amino acids in the protein can be attached to a label that uniquely identifies the type of amino acid. In this configuration, the change in signal that identifies the amino acid can be loss of signal from the respective label. For example, lysines can be attached to a distinguishable label such that loss of the label indicates removal of a lysine. Alternatively or additionally, other amino acid types can be attached to other labels that are mutually distinguishable from lysine and from each other. Exemplary compositions and techniques that can be used to remove amino acids from a protein and detect signal changes are those set forth in Swaminathan et al., Nature Biotech. 36:1076-1082 (2018); or U.S. Pat. Nos. 9,625,469 or 10,545,153, each of which is incorporated herein by reference.

In a second configuration of a cyclical protein detection assay, a terminal amino acid of a protein can be recognized by an affinity reagent that is specific for the terminal amino acid or specific for a label moiety that is present on the terminal amino acid. The affinity reagent can be detected on an array, for example, due to a label on the affinity reagent. Optionally, the label is a nucleic acid barcode sequence that is added to a primer nucleic acid upon formation of a complex. For example, a barcode can be added to the primer via ligation of an oligonucleotide having the barcode sequence or polymerase extension directed by a template that encodes the barcode sequence. The formation of the complex and identity of the terminal amino acid can be determined by decoding the barcode sequence. Multiple cycles can produce a series of barcodes that can be detected, for example, using a nucleic acid sequencing technique. Exemplary affinity agents and detection methods are set forth in US Pat. App. Pub. No. 2019/0145982 A1; 2020/0348308 A1; or 2020/0348307 A1, each of which is incorporated herein by reference.

Cyclical removal of terminal amino acids from a protein can be carried out using an Edman-type sequencing reaction in which a phenyl isothiocyanate reacts with a N-terminal amino group under mildly alkaline conditions (e.g. about pH 8) to form a cyclical phenylthiocarbamoyl Edman complex derivative. The phenyl isothiocyanate may be substituted or unsubstituted with one or more functional groups, linker groups, or linker groups containing functional groups. Many variations of Edman-type degradation have been described and may be used including, for example, a one-step removal of an N-terminal amino acid using alkaline conditions (Chang, J. Y., FEBS LETTS., 1978, 91(1), 63-68).

Edman-type processes can be carried out in a multiplex format to detect, characterize or identify a plurality of proteins. A method of detecting a protein can include steps of (i) exposing a terminal amino acid on a protein at an address of an array; (ii) binding an affinity agent to the terminal amino acid, where the affinity agent includes a nucleic acid tag, and where a primer nucleic acid is present at the address; (iii) extending the primer nucleic acid, thereby producing an extended primer having a copy of the tag; and (iv) detecting the tag of the extended primer. The terminal amino acid can be exposed, for example, by removal of one or more amino acids from the amino terminus or carboxyl terminus of the protein. Steps (i) through (iv) can be repeated to produce a series of tags that is indicative of the sequence for the protein. The method can be applied to a plurality of proteins on the array and in parallel. Whatever the plexity, the extending of the primer can be carried out, for example, by polymerase-based extension of the primer, using the nucleic acid tag as a template. Alternatively, the extending of the primer can be carried out, for example, by ligase- or chemical-based ligation of the primer to a nucleic acid that is hybridized to the nucleic acid tag. The nucleic acid tag can be detected via hybridization to nucleic acid probes (e.g. in an array), amplification-based detections (e.g. PCR-based detection, or rolling circle amplification-based detection) or nucleic acid sequencing (e.g. cyclical reversible terminator methods, nanopore methods, or single molecule, real time detection methods). Exemplary methods that can be used for detecting proteins using nucleic acid tags are set forth in US Pat. App. Pub. No. 2019/0145982 A1; 2020/0348308 A1; or 2020/0348307 A1, each of which is incorporated herein by reference.

A protein assay can optionally detect a protein based on its enzymatic or biological activity. For example, a protein can be contacted with a reactant that is converted to a detectable product by an enzymatic activity of the protein. In other assay formats, a first protein having a known enzymatic function can be contacted with a second protein to determine if the second protein changes the enzymatic function of the first protein. As such, the first protein serves as a reporter system for detection of the second protein. Exemplary changes that can be observed include, but are not limited to, activation of the enzymatic function, inhibition of the enzymatic function, attenuation of the enzymatic function, degradation of the first protein or competition for a reactant or cofactor used by the first protein. Proteins can be categorized by type according to detectable characteristics of their activity such as ability or inability to modify a particular reactant, ability or inability to catalyze a particular reaction, ability or inability to produce a particular reaction product, sensitivity or inertness to a particular enzyme inhibitor, sensitivity or inertness to a particular enzyme activator, ability or inability to catalyze a reaction at a particular rate, ability or inability to catalyze a reaction in a particular condition such as at a particular reagent concentration or a combination of the foregoing. Proteins can also be detected based on their binding interactions with other molecules such as nucleic acids, nucleotides, other proteins, protein domains, metabolites, hormones, vitamins, small molecules that participate in biological signal transduction pathways, biological receptors or the like. Proteins can be categorized by type according to binding characteristics. For example, a protein that participates in a signal transduction pathway can be identified as a particular candidate protein by detecting binding to a second protein that is known to be a binding partner for the candidate protein in the pathway.

What is claimed is:

1. A method of detecting proteoforms, comprising
(a) providing a first aliquot from a biological sample and a second aliquot from the biological sample, wherein a plurality of different proteoforms that are present in the first aliquot is also present in the second aliquot, wherein the proteoforms comprise post-translationally modified amino acids and standard amino acids;
(b) treating proteoforms of the first aliquot by:
(i) chemically modifying the standard amino acids, thereby forming artificially modified amino acids,
(ii) then removing post-translational modifications from the post-translationally modified amino acids, thereby forming standard amino acids, and
(iii) then detecting proteoforms of the first aliquot;
(c) detecting proteoforms of the second aliquot; and
(d) identifying the presence of a post-translational modification in a proteoform of the biological sample based on differential detection of a proteoform in the first aliquot and in the second aliquot.

2. The method of claim 1, wherein the detecting of the proteoforms of the first aliquot comprises performing a binding assay between an affinity reagent and the proteoforms of the first aliquot.

3. The method of claim 2, wherein the detecting of the proteoforms of the second aliquot comprises performing a binding assay between the affinity reagent and the proteoforms of the second aliquot.

4. The method of claim 3, wherein the affinity reagent selectively recognizes a standard version of an amino acid compared to a post-translationally modified version of the amino acid and compared to an artificially modified version of the amino acid.

5. The method of claim 3, wherein the affinity reagent selectively recognizes a post-translationally modified version of an amino acid compared to the standard version of the amino acid and compared to an artificially modified version of the amino acid.

6. The method of claim 3, wherein the affinity reagent selectively recognizes an artificially modified version of an amino acid compared to a post-translationally modified version of the amino acid and compared to the standard version of the amino acid.

7. The method of claim 2, further comprising attaching the proteoforms of the first aliquot to unique identifiers via artificially modified amino acids formed in step (b)(i).

8. The method of claim 7, wherein step (c) comprises treating proteoforms of the second aliquot by:
(i) chemically modifying the standard amino acids, thereby forming artificially modified amino acids,
(ii) attaching the proteoforms of the second aliquot to unique identifiers via artificially modified amino acids formed in step (c)(i), and
(iii) performing the binding assay between the affinity reagent and the proteoforms of the second aliquot.

9. The method of claim 2, further comprising attaching the proteoforms of the first aliquot to unique identifiers via standard amino acids formed in step (b)(ii).

10. The method of claim 9, wherein step (c) comprises: treating proteoforms of the second aliquot by:
(i) attaching the proteoforms of the second aliquot to unique identifiers via standard amino acids, and
(ii) performing the binding assay between the affinity reagent and the proteoforms of the second aliquot.

11. The method of claim 1, wherein step (b) further comprises attaching individual proteoforms of the first aliquot to unique identifiers via a standard amino acid formed in step (b)(ii).

12. The method of claim 11, wherein step (c) further comprises attaching individual proteoforms of the second aliquot to unique identifiers via a standard amino acid.

13. The method of claim 1, wherein step (b)(i) comprises chemically modifying the standard amino acids with blocking moieties, thereby forming blocked amino acids.

14. The method of claim 13, wherein the blocked amino acids are inert to the removing of the post-translational modifications from the post-translationally modified amino acids.

15. The method of claim 13, wherein the blocked amino acids are inert to reaction with unique identifiers, the reaction resulting in attachment of standard amino acids to the unique identifiers.

16. The method of claim 1, wherein the detecting of proteoforms of the first aliquot comprises detecting standard amino acids and artificially modified amino acids of the proteoforms of the first aliquot.

17. The method of claim 16, wherein the detecting of proteoforms of the second aliquot comprises detecting standard amino acids and artificially modified amino acids of the proteoforms of the second aliquot.

18. The method of claim 16, wherein the detecting of the standard amino acids comprises binding the standard amino acids to affinity reagents that selectively bind the standard amino acids compared to the artificially modified amino acids and compared to the post-translationally modified amino acids.

19. The method of claim 16 wherein the detecting of the artificially modified amino acids comprises binding the artificially modified amino acids to affinity reagents that selectively bind the artificially modified amino acids compared to the standard amino acids and compared to the post-translationally modified amino acids.

20. A method of labeling proteoforms, comprising
(a) providing a first aliquot from a biological sample and a second aliquot from the biological sample, wherein a plurality of different proteoforms that are present in the first aliquot is also present in the second aliquot, wherein the proteoforms comprise post-translationally modified amino acids and standard amino acids;
(b) treating proteoforms of the first aliquot by:
(i) chemically modifying the standard amino acids, thereby forming artificially modified amino acids,
(ii) then removing post-translational modifications from the post-translationally modified amino acids, thereby forming standard amino acids,
(iii) then attaching the proteoforms of the first aliquot to a first set of unique identifiers; and
(c) treating proteoforms of the second aliquot by attaching the proteoforms of the second aliquot to a second set of unique identifiers.

21. A method of detecting a proteoform, comprising
(a) contacting an array of proteoforms with a first affinity reagent and a second affinity reagent, thereby forming a complex comprising the first affinity reagent bound to a post-translationally modified amino acid in a proteoform at an address of the array and the complex further comprising the second affinity reagent bound to a sequence of standard amino acids in the proteoform,
wherein the first affinity reagent is attached to a first nucleic acid and the second affinity reagent is attached to a second nucleic acid;
(b) contacting the complex with a splint nucleic acid, wherein a first nucleotide sequence region of the splint nucleic acid hybridizes to the first nucleic acid and a second nucleotide sequence region of the splint nucleic acid hybridizes to the second nucleic acid;
(c) detecting hybridization of the first and second nucleic acids to the splint nucleic acid, thereby detecting the proteoform at the address.

22. A method, comprising
(a) contacting an array of proteoforms with a multivalent affinity reagent, thereby forming a complex comprising a first affinity moiety of the multivalent affinity reagent bound to a post-translationally modified amino acid of a proteoform at an address of the array, the complex further comprising a second affinity moiety of the multivalent affinity reagent bound to a sequence of standard amino acids in the proteoform; and
(b) detecting the complex at the address.


SEQUENCE LISTING (1 sequence)

SEQ ID NO: 1 - protein, 17 aa, synthetic construct
  REGION 1..17: note = NTSNE STDVT KGDSK NA
NTSNESTDVTKGDSKNA